(19) United States
(12) Patent Application Publication
(10) Pub. No.: US 2026/0102511 A1
(43) Pub. Date: Apr. 16, 2026

(54) THIOL-MODIFIED POLYMERIC NANOPARTICLES FOR TUMOR-SPECIFIC GENE DELIVERY

(71) Applicant: UNIVERSITY OF VIRGINIA PATENT FOUNDATION, Charlottesville, VA (US)

(72) Inventors: Anna Debski, Charlottesville, VA (US); Richard J. Price, Charlottesville, VA (US)

(21) Appl. No.: 19/358,244

(22) Filed: Oct. 14, 2025

Related U.S. Application Data
(60) Provisional application No. 63/706,997, filed on Oct. 14, 2024.

(51) Int. Cl. A61K 48/00; A61K 38/19

(52) U.S. Cl. CPC A61K 48/0041; A61K 38/195; A61K 48/0058

(57) ABSTRACT

Thiol decorated polymeric nanoparticles, and methods of their use, are described herein. The thiol decorated nanoparticles are useful for treating cancers or disorders characterized by the presence of cells enriched with exofacial thiols. Exemplary nanoparticles comprise a PEI-g-PEG-SH graft copolymer and a branched PEI polymer. Exemplary nanoparticles are loaded with therapeutic agents.

### CROSS-REFERENCE TO RELATED APPLICATION(S)

This application claims the benefit of and priority to U.S. Provisional Application No. 63/706,997 filed on Oct. 14, 2024. The content of which is incorporated by reference in its entirety.

### STATEMENT REGARDING FEDERALLY SPONSORED RESEARCH

This invention was made with government support under EB030744 awarded by the National Institutes of Health. The government has certain rights in the invention.

### BACKGROUND

Glioblastoma (GBM) is a devastatingly difficult disease to treat. Despite being the most commonly diagnosed malignant tumor in the central nervous system, prognosis remains poor with a 2-year survival rate of ˜30% and almost complete mortality. Despite both extensive research and medical efforts, the median survival for GBM has only increased 4 months since 2005, highlighting the need for more innovative and effective therapies. A major challenge in treating GBM is the blood-brain barrier (BBB), which restricts passage of approximately 98% of small molecules, including anti-cancer drugs, into the brain, reducing efficacy and necessitating high doses that increase toxicity. In GBM, rapid tumor growth and angiogenesis disrupts the BBB, forming a leaky blood-tumor barrier (BTB) with pathological features like increased permeability, drug efflux pump overexpression, immunosuppressive signaling, and dysregulated protein synthesis. While these abnormalities drive tumor progression and therapy resistance, they also present unique opportunities for targeted treatment strategies.

### SUMMARY

In an aspect, provided herein is a method comprising administering to a subject in need thereof, a polymeric nanoparticle comprising free thiol groups. The nanoparticle may comprise thiol-functionalized polyethylene glycol (PEG-SH). The nanoparticle may comprise polyethyleneimine (PEI), poly(beta-amino ester) (PBAE), or bioreducible PEI (rPEI). The nanoparticle may comprise a PEI-g-PEG-SH graft copolymer and a branched PEI polymer.

The nanoparticle may be loaded with a therapeutic nucleic acid operably linked to a promoter. The promoter may be an endothelial cell-specific promoter. The promoter may be a CD144 promoter.

The therapeutic nucleic acid may encode a chemokine. The chemokine may be CXCL9.

The nanoparticle may be administered intravenously.

The method may further comprise administering microbubbles and focused ultrasound to the subject. The nanoparticle may be administered between about 30 minutes before and about 30 minutes after the FUS is administered.

The method may further comprise administering an immunotherapy agent. The immunotherapy agent may comprise an anti-PD-1 antibody.

The subject may have a cancer. In embodiments, the cancer is a glioblastoma.

In another aspect, provided herein is a polymeric nanoparticle comprising free thiol groups, wherein the nanoparticle is loaded with a therapeutic nucleic acid operably linked to a promoter. The nanoparticle may comprise thiol-functionalized polyethylene glycol (PEG-SH). The nanoparticle may comprise polyethyleneimine (PEI), poly(beta-amino ester) (PBAE), or bioreducible PEI (rPEI). The nanoparticle may comprise a PEI-g-PEG-SH graft copolymer and a branched PEI polymer.

### BRIEF DESCRIPTION OF THE DRAWINGS

The patent or patent application file contains at least one drawing in color. Copies of this patent or patent application publication with color drawings will be provided by the Office upon request and payment of the necessary fee.

The present disclosure will be better understood and features, aspects, and advantages other than those set forth above will become apparent when consideration is given to the following detailed description thereof. Such detailed description makes reference to the following drawings.

FIGS. 1A-1W. Characterization of SH-NP Transfection Tropism. (A) Ellman's reagent analysis of exofacial thiols in non-tumor cells (bEnd.3), tumor cells (GL261-Luc2), and in bEnd.3 cells conditioned with GL261-Luc2 media (n=3; means±SD; ***p<0.001, ****p<0.0001, unpaired t-test). (B) Characterization of physiochemical properties of SH-NPs. (C) Quantification of ex vivo imaging of off-target organs following CED and IV injection of E2-Crimson plasmid-loaded SH-NPs (n=5, unpaired t-test). Characterization of percent of tumor cells (D), endothelial cells (E), astrocytes (F), pericytes (G), and leukocytes (H) following IV injection, CED, or FUS+IV administration of SH-NPs (IV: n=4, CED: n=5. FUS+IV: n=9; one-way analysis of variance (ANOVA) followed by Tukey's multiple comparison's test, ****p<0.0001). Characterization of MFI of transfected cells of tumor cells (I), endothelial cells (J), astrocytes (K), pericytes (L), and leukocytes (M) following IV injection, CED, or FUS+IV administration of SH-NPs (IV: n=4, CED: n=5. FUS+IV: n=9; ANOVA followed by Tukey's multiple comparison's test, ****p<0.0001). Analysis of influence timing of SH-NP delivery relative to FUS treatment on percentage of cells and MFI of transfected cells, respectively, following FUS of tumor cells (N, S), endothelial cells (O, T), astrocytes (P, U), pericytes (Q, V) and leukocytes (R, W; +15: n=3, 0: n=3, −15: n=3; ANOVA followed by Tukey's multiple comparison's test.

FIGS. 2A-2G. Sonoselective Transfection of GBM Endothelium. (A) Representative flow cytometry plots for H2-K^{k} transfected cells in the TME following FUS+IV of CD-144-H2-K^{k} plasmid-loaded SH-NPs. (B) Quantification of flow cytometry analysis of transfected cells in the TME (n=5; one-way ANOVA followed by Dunnett's multiple comparisons tests versus EC, ****p<0.0001). Confocal images of GL261-Luc2 tumors following FUS-mediated delivery of SH-NPs bearing CMV-H2-K^{k} (C) and CD144-H2-K^{k} plasmids (D; red: H2-K^{k}, green: CD31, blue: DAPI, scale bars: 100 μm, yellow arrows: H2-K^{k} transfection outside of endothelial cells, white arrows: co-localization between CD31 and H2-K^{k}). (E) Quantification of correlation between CD31⁺ and H2-K^{k+} area (n=3, unpaired t-test *p<0.05). (F) Average PNP over 3 sonication points per mouse following FUS-mediated delivery of CMV-H2-K^{k} and CD144-H2-K^{k} plasmids encapsulated in SH-NPs (n=5, unpaired t-test). (G) Applied PNP over time during PCD-modulated FUS treatments. Each line represents the average applied PNP over 3 sonication points per 5 mice±SEM (n=5, unpaired t-test).

FIGS. 3A-3S. Sonoselective Transfection of GL261-Luc2 Tumor Endothelium for Improved Immune Cell Infiltration. (A) Quantification of flow cytometry analysis of CXCL9 chemokine MFI following transfection of bEnd.3 cells with CXCL9 and CXCL10 plasmids compared to transfected control (CTRL) cells (n=6; one-way ANOVA followed by Dunnett's multiple comparisons tests versus CTRL, ****p<0.0001). (B) Quantification of flow cytometry analysis of CXCL10 chemokine MFI following transfection of bEnd.3 cells with CXCL9 and CXCL10 plasmids compared to transfected control (CTRL) cells (n=6; one-way ANOVA followed by Dunnett's multiple comparisons tests versus CTRL, *p<0.05). (C) Quantification of flow cytometry analysis of T cell recruitment following co-culture with bEnd.3 cells transfected with CXCL9 and CXCL10 chemokines (n=4, one-way ANOVA followed by Dunnett's multiple comparisons tests versus CTRL, ***p<0.001, ****p<0.0001). (D) Quantification of T cell counts in Transwells® following co-culture with bEnd.3 cells transfected with CXCL9 and CXCL10 chemokines (n=6, one-way ANOVA followed by Dunnett's multiple comparisons tests versus CTRL). (E) CXCL9 and CXCL10 plasmid vector. (F) Timeline of sonoselective transfection of SH-NPs encapsulating CXCL9 plasmids to quantify CXCL9 expression and profile immune cell infiltration. Percentage (G) and MFI (H) of CXCL9⁺ endothelial cells (n=3, unpaired t-test, *p<0.05). Percentage (I) and MFI (J) of CXCL9⁺ tumor cells (n=3, unpaired t-test). (K) Number of CD45+ cells in all treatment groups (n=5, one-way ANOVA followed by Dunnett's multiple comparisons tests). Cell counts and percentages, respectively, of NK cells (L, P), cytotoxic T cells (M, Q), Tregs (N, R), and helper T cells (O, S) following sonoselective transfection of SH-NPs encapsulating CXCL9 plasmids (n=5, ANOVA followed by Tukey's multiple comparison's test, *p<0.05, **p<0.01, ***p<0.001).

FIGS. 4A-4C. Combination Therapy of Sonoselective Transfection of CXCL9 and aPD1. (A) Schematic of timeline of combination therapy. (B) Kaplan-Meier curve depicting overall survival (n=6, cox regression analysis). (C) In vivo bioluminescence images of GL261-Luc2 tumors.

FIG. 5. Flow Cytometry Gating Strategy. Gating strategy used for analyzing flow cytometry data following SH-NP delivery of reporter plasmids.

FIGS. 6A-6F. Representative Flow Plots from Flow Cytometry Transfection Analysis of SH-NPs Encapsulating CMV-H2-K^{k}. Flow plots representing percentage of cells transfected (A) and MFI of transfected cells (B) following IV injection. Flow plots representing percentage of cells transfected (C) and MFI of transfected cells (D) following CED. Flow plots representing percentage of cells transfected (E) and MFI of transfected cells (F) following FUS+IV.

FIG. 7. Acoustic Emissions Signatures from PCD-Modulated Experiments. (A) Acoustic emissions signatures following FUS-mediated delivery of CMV-H2-k^{k}. (B) Acoustic emissions signatures following FUS-mediated delivery of CD144-H2-k^{k}.

FIGS. 8A-8D. In Vitro Gating Strategy. (A) Gating strategy for in vitro flow cytometry analysis. Representative flow plot of CXCL9 MFI (B) and CXCL10 MFI (C). Representative flow plot of quantification of T cell recruitment from co-culture assay marked by CD45⁺ (D).

FIG. 9. Flow Cytometry Gating Strategy for In Vivo CXCL9 Quantification. Gating strategy used for analyzing flow cytometry data quantifying CXCL9 and CXCL9 in vivo.

FIG. 10. Gating Strategy Profiling Immune Cell Infiltration in GBM Tumors. Gating strategy and representative flow plots of immune cell populations after sonoselective delivery of CXCL9 plasmid-bearing SH-NPs to the tumor endothelium.

### DETAILED DESCRIPTION

Glioblastoma is a highly aggressive brain cancer, and its treatment is challenged by the blood-brain and blood-tumor barriers, impeding effective therapeutic delivery. As GBM progresses, disruptions in cellular processes create a tumor microenvironment (TME) that not only drives tumor growth and therapy resistance but also presents unique targets for gene therapy. Notably, cells within the TME are enriched with exofacial thiols, or free thiols exposed on the cell surface. Leveraging this characteristic, the inventors developed polymeric nanoparticles functionalized with free thiol groups (SH-NPs) to facilitate tumor targeting and transfection. They used focused ultrasound to deploy SH-NPs for precise transfection of the tumor endothelium, and demonstrated the therapeutic benefits of this platform using a GBM murine model to transfect chemokine-encoding plasmids for enhanced immune cell infiltration into tumors. By achieving highly specific endothelial cell targeting, this platform enables screening of a broad range of therapeutic targets within the endothelium.

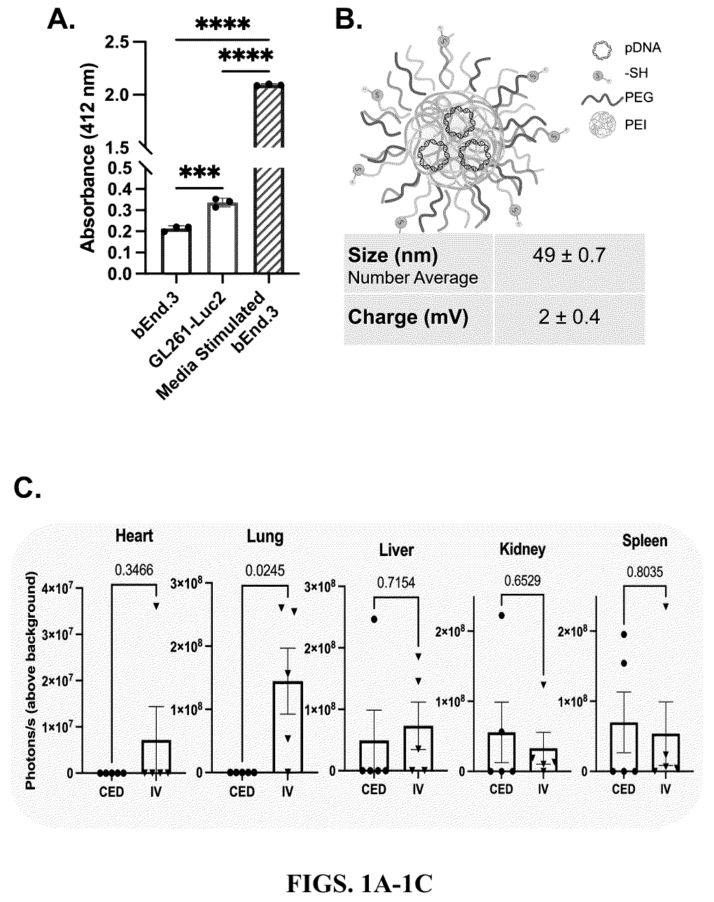

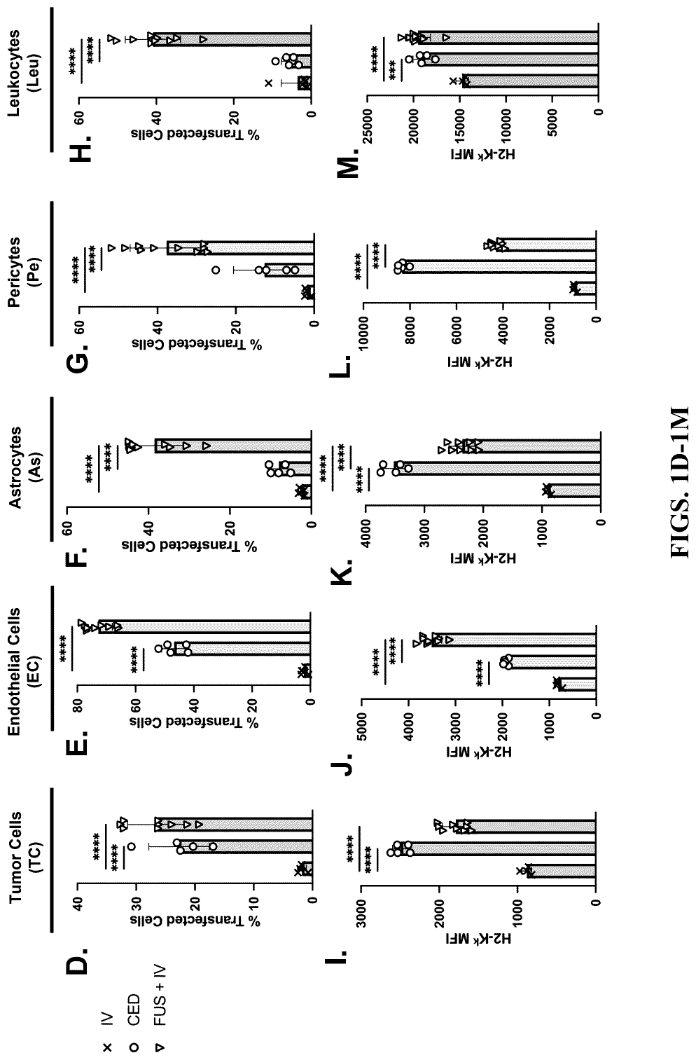

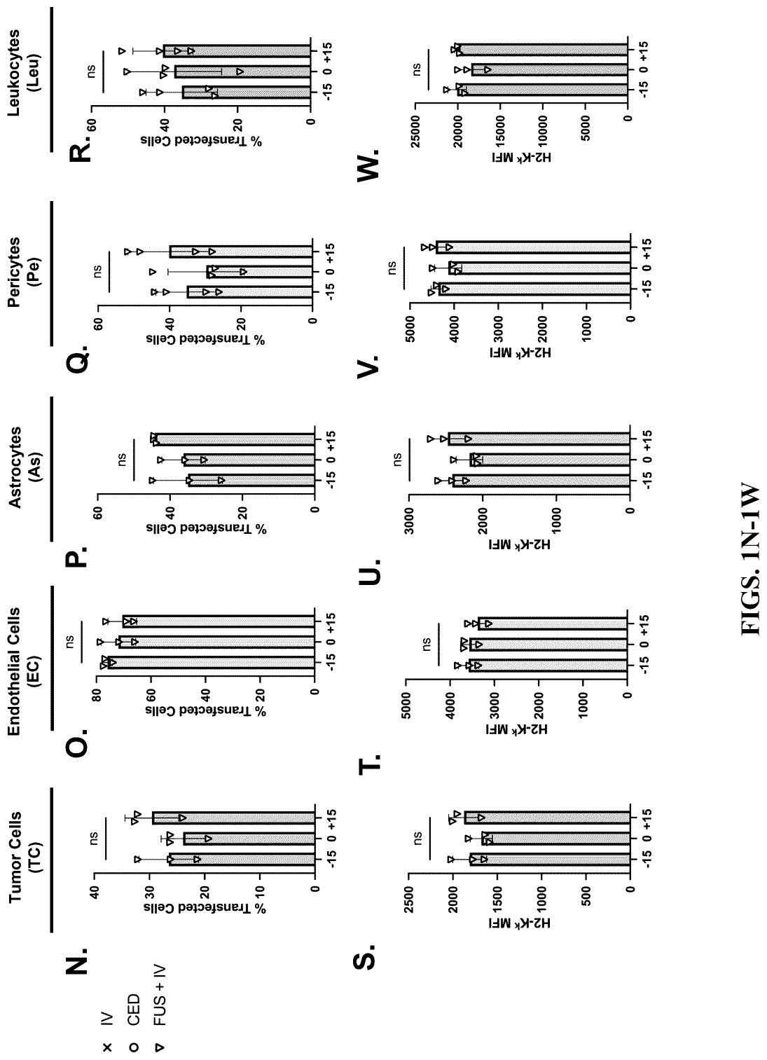

In a first aspect, provided herein is a method comprising administering to a subject in need thereof, a polymeric nanoparticle comprising free thiol groups. The thiol groups decorate the surface of the nanoparticle, as illustrated at FIG. 1B.

The term “nanoparticle,” as used herein, refers to a particle having at least one dimension, e.g. a diameter, in the range of about 1 nm to about 1000 nm, including any integer or fractional integer value between 1 nm and 1000 nm (including about 1, 2, 5, 10, 20, 50, 60, 70, 80, 90, 100, 200, 500, 1000 nm, etc.). In exemplary embodiments, the nanoparticle is about 50 nm in diameter.

Nanoparticles suitable for use in the presently disclosed compositions and methods may exist in a variety of shapes, including, but not limited to, spheroids, rods, disks, pyramids, cubes, cylinders, nanohelixes, nanosprings, nanorings, rod-shaped nanoparticles, arrow-shaped nanoparticles, teardrop-shaped nanoparticles, tetrapod-shaped nanoparticles, prism-shaped nanoparticles, and a plurality of other geometric and non-geometric shapes. In embodiments, the nanoparticles have a spherical shape.

Polymer-based nanoparticles typically are formed from biocompatible and biodegradable block co-polymers of different hydrophobicity. (See Chan et al., In: Cancer Nanotechnology; Grobmyer SR, Moudgil BM, editors. Vol. 624. Humana Press; 2010. pp. 163-175)). These copolymers spontaneously assemble into a core-shell micelle formation in an aqueous environment. (See Torchilin, V.P., Pharm Res., 24:1-16 (2007)). Polymeric nanoparticles have been formulated to encapsulate hydrophilic and/or hydrophobic small drug molecules, as well proteins and nucleic acid macromolecules. (See Wang et al., Expert Opinion on Biological Therapy, 8:1063-1070 (2008)).

The polymeric nanoparticles described herein may comprise at least one of thiol-functionalized polyethylene glycol (PEG-SH); and polyethyleneimine (PEI), poly(beta-amino ester) (PBAE), or bioreducible PEI (rPEI). In embodiments, the polymeric nanoparticle comprises a) a thiol-functionalized polyethylene glycol (PEG-SH) and b) at least one of a PEI, an rPEI, and a PBAE. In embodiments, the polymeric nanoparticle comprises a thiol functionalized PEG and a PEI. In other embodiments, the polymeric nanoparticle comprises a thiol-functionalized PEG and an rPEI. In other embodiments, the polymeric nanoparticle comprises a thiol-functionalized PBAE. The thiol-functionalized PBAE may comprise a PBAE that is conjugated to thiol-modified PEG groups.

In exemplary embodiments, the nanoparticle comprises a) a PEI-g-PEG-SH graft copolymer, wherein the PEG-SH polymers are grafted onto a branched PEI polymer; and b) an additional, non-PEGylated branched PEI polymer. The PEG-SH may have a molecular weight of between about 2 and about 5 kDa. In embodiments, the PEG-SH has a molecular weight of about 5 kDa. The PEI in the graft copolymer and the branched PEI polymer may have a molecular weight of between about 2 and about 25 kDa. In embodiments, the PEI has a molecular weight of about 25 kDa. The nanoparticle may comprise between about 3:1 to 1:1 PEI-g-PEG-SH to PEI ratio. In embodiments, the NP has a PEI-g-PEG-SH:PEI ratio of 1:1. The nanoparticle may be formulated at an N (amine from polymer):P (phosphate groups from nucleic acid) ratio of between about 8:1 to 1:1. In embodiments, the nanoparticle comprises an N:P ratio of about 6:1.

In exemplary embodiments, the nanoparticle may also comprise a) a PEI-g-PEG-SH graft copolymer, wherein the PEG-SH polymers are grafted onto a branched PEI polymer; and b) an additional, non-PEGylated bioreducible linear PEI polymer (rPEI). The PEG-SH may have a molecular weight of between about 2 and about 5 kDa. In embodiments, the PEG-SH has a molecular weight of about 5 kDa. The PEI in the graft copolymer and the rPEI polymer may have a molecular weight of between about 2 and about 25 kDa. In embodiments, the graft copolymer PEI has a molecular weight of about 25 kDa and the bioreducible linear PEI has a molecular weight of about 2.5 kDa. The nanoparticle may comprise between about 3:1 to 1:1 PEI-g-PEG-SH to rPEI ratio. In embodiments, the NP has a PEI-g-PEG-SH:rPEI ratio of 1:1. The nanoparticle may be formulated at an N (amine from polymer):P (phosphate groups from nucleic acid) ratio of between about 20:1 to 1:1. In embodiments, the nanoparticle comprises an N:P ratio of about 18:1.

The terms “patient”, “subject”, and “individual” refer to all animals, including mammals, e.g., a human or a non-human mammal, who are prone to or suffering from the indicated disease or disorder, or who are treated with compositions and/or methods described herein. The subject may be a mammal in need of treatment for a cancer. The subject may be a human.

A “subject in need thereof” refers to having or at risk of having a disorder characterized by the presence of cells enriched with exofacial thiols. In embodiments, the subject has a cancer. As used herein, “cancer” refers to diseases in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that begins in blood-forming tissue, such as the bone marrow, and causes too many abnormal blood cells to be made. Lymphoma and multiple myeloma are cancers that begin in the cells of the immune system. Central nervous system cancers are cancers that begin in the tissues of the brain and spinal cord. In embodiments, the cancer comprises a solid tumor.

In embodiments, the subject is diagnosed with, or suspected of having a brain cancer or a brain tumor. The brain cancer or tumor may be of any type or origin, and may be present in a pediatric or adult subject. The brain tumor may be glioblastoma (GBM), an embryonal tumor, a neuroblastoma, a glioma, an ependymoma, an optic nerve glioma, a craniopharyngioma, meningioma, pituitary tumor, vestibular schwannoma, medulloblastoma, choroid plexus tumor, pineal tumor, chordoma, chondrosarcoma, olfactory neuroblastoma, astrocytoma, ependymal tumor, hemangiopericytoma a germ cell tumor, oligodendroglioma, brain metastases or a ganglioglioma. In exemplary embodiments, the subject has glioblastoma.

The nanoparticle may be loaded with a plasmid comprising a therapeutic nucleic acid operably linked to a promoter. The term “therapeutic nucleic acid” refers to a nucleic acid that has the ability to treat or prevent a disease or disorder, or that encodes a protein that has the ability to treat or prevent a disease or disorder. In embodiments, the therapeutic nucleic acid encodes a protein that recruits an immune cell to the location to which the nanoparticle has been administered, e.g. a tumor or tumor microenvironment. In embodiments, the therapeutic nucleic acid encodes a chemokine. Immune cell recruiting chemokines include, but are not limited to, CXCL8, CXCL9, CXCL10, CXCL11, CXCL12, CCL2, CCL3, CCL5, and CCL20. In exemplary embodiments, the chemokine is CXCL9.

The terms “nucleic acid” and “polynucleotide” refer to a nucleotide, oligonucleotide, polynucleotide (which terms may be used interchangeably), or any fragment thereof. A “polynucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. These terms refer only to the primary structure of the molecule. Thus, these terms include double-and single-stranded DNA, as well as double-and single-stranded RNA. For use in the present methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand). Nucleic acids include, but are not limited to, deoxyribonucleic acid (DNA), ribonucleic acid (RNA), DNA/RNA hybrids, pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (geRNA), guide RNA, tracRNA, crRNA, sgRNA, plus strand RNA (RNA(+)), minus strand RNA (RNA(−)), synthetic RNA, genomic DNA (gDNA), PCR amplified DNA, complementary DNA (cDNA), synthetic DNA, or recombinant DNA.

A “recombinant nucleic acid sequence” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence.

As used herein, the term “construct” refers to a recombinant polynucleotide, i.e., a polynucleotide that was formed artificially by combining at least two polynucleotide components from different sources (natural or synthetic). Constructs can be generated using conventional recombinant DNA methods. Constructs described herein may comprise at least one therapeutic nucleic acid operably linked to a promoter. As used herein, the term “promoter” refers to a DNA sequence that regulates the transcription of a polynucleotide. Typically, a promoter is a regulatory region that is capable of binding RNA polymerase and initiating transcription of a downstream sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA segments. It is understood by those skilled in the art that different promoters may direct the expression of a gene in different tissues or cell types, at different stages of development, or in response to different environmental conditions. A promoter is “operably linked” to a polynucleotide if the promoter is connected to the polynucleotide such that it may affect transcription of the polynucleotide.

The promoter used in the constructs described herein may be a heterologous promoter (i.e., a promoter that is not naturally associated with the encoded protein), an endogenous promoter (i.e., a promoter that is naturally associated with the encoded protein), or a synthetic promoter that is designed to function in a desired manner in a particular host cell. Suitable promoters for use with the present invention include, but are not limited to, constitutive, inducible, temporally regulated, developmentally regulated, chemically regulated, tissue-preferred, and tissue-specific promoters.

In embodiments, the promoter is an endothelial cell-specific promoter. In exemplary embodiments, the promoter is a CD144 promoter. Other endothelial cell-specific promoters include, but are not limited to, a CDH5 promoter; a PEC AMI promoter; an ERG promoter; a TEK promoter; a KDR promoter; a SOX17 promoter; a VWF promoter; an ESMI promoter; a PR0X1 promoter; a VEGFR3 promoter, a FLT4 promoter; a PDPN promoter; and a LYVE1 promoter.

In embodiments, the promoter is a cancer cell-specific promoter. Cancer cell-specific promoters include, but are not limited to, an alpha-fetoprotein (AFP) promoter, a VWA1 promoter, a Cholecystokinin-A receptor (CCKAR) promoter, a carcinoembryonic antigen (CEA) promoter, a C-erbB2/neu promoter, a cyclooxygenase (COX-2) promoter, a CXCR4 promoter, a E2F promoter, a human epididymis protein 4 (HE4), a L-plastin (LP) promoter, a MUC1 promoter, a prostate-specific antigen (PSA) promoter, a Survivin promoter, a tyrosinase-related protein 1 (TRP1) promoter, an EGFR promoter, and a tyrosinase promoter.

The terms “plasmid” and “vector,” as used herein, refer to a nucleic acid molecule capable of propagating another nucleic acid to which it is linked. The term includes the vector as a self-replicating nucleic acid structure as well as the vector incorporated into the genome of a host cell into which it has been introduced. An “expression vector” or “recombinant expression vector” is a vector that is capable of directing the expression of exogenous genes. In general, expression vectors of utility in recombinant DNA techniques are often in the form of plasmids. Expression vectors include all those known in the art, including, without limitation, a yeast artificial chromosome, bacterial plasmid (e.g., naked or contained in liposomes), phagemid, shuttle vector, cosmid, virus (e.g., Sendai viruses, lentiviruses, retroviruses, adenoviruses, and adeno-associated viruses), chromosome, mitochondrial DNA, plastid DNA, and nucleic acid fragment. Generally, an expression vector comprises sufficient cis-acting elements for expression; other elements for expression can be supplied by the host cell or in an in vitro expression system.

In embodiments, the nanoparticle is loaded with a small molecule therapeutic. Small-molecule therapeutics/drugs are typically comprised of 20 to 100 atoms and have a molecular mass of less than 1000 g/mol or 1 kilodalton [kDa]. The small molecule therapeutic may be a chemotherapeutic or an anti-cancer drug.

As used herein, the term “administering” an agent, such as a therapeutic agent to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either intravenous injection, the parenteral or oral route, intramuscular injection, subcutaneous/intradermal injection, intrathecal administration, buccal administration, transdermal delivery, topical administration, and administration by the intranasal or respiratory tract route. In exemplary embodiments, the nanoparticle is administered intravenously.

The method may further comprise administering a microbubble composition and focused ultrasound (FUS) to the subject. Microbubbles (MBs) in the field of medical diagnostics are typically used as contrast agents for ultrasound imaging. Smaller than one hundredth of a millimeter in diameter, but larger than one micrometer, a MB is comprised of a gas encapsulated by a shell. Shell materials may comprise one or more of lipids, albumins, proteins, etc. The gas is typically air or a perfluorocarbon. The gas may be any useful gas that can be used safely for the described methods. A microbubble composition may comprise a homogenous or heterogenous population of microbubbles. For example, each microbubble in the microbubble composition may be of the same or similar size, gas, shell, etc. In other examples, microbubbles in the microbubble composition may have different sizes, gases, shells, etc.

“Ultrasound” is sound with frequencies greater than 20 kilohertz (kHz) generated by an ultrasonic transducer. The ultrasound may be administered with a 1.1 MHz single element transducer. In a subject having a solid tumor, the ultrasound may be applied directly to the tumor. In exemplary embodiments, the ultrasound is applied at 3 sonication points per tumor. The ultrasound may be administered with pulses of between about 10 microseconds and about 880 milliseconds (ms) with pulsing intervals that allow replenishment of microbubbles between cavitation pulses. The ultrasound may be administered with about 60 pulses of about 10 ms per pulse.

The polymeric nanoparticle may be administered between about 30 minutes before and about 30 minutes after the FUS is administered. In embodiments, the nanoparticle is administered between about 15 minutes before and about 15 minutes after the FUS is administered. In embodiments, the nanoparticle is administered at about the same time as the administering of the FUS.

The method may comprise administering the nanoparticle by convection-enhanced delivery (CED). Convection-enhanced delivery (CED) is a technique that bypasses the blood-brain barrier (BBB) to deliver drugs directly to the brain or other tissues by using a surgically placed catheter connected to an infusion pump to generate a pressure gradient, forcing the therapeutic agent into the extracellular matrix.

The method may further comprise administering to the subject an additional cancer treatment. Additional cancer treatments include, but are not limited to chemotherapy, radiation, bone marrow transplant, surgery and immunotherapy.

The immunotherapy agent may comprise an immune checkpoint blockade antibody. Immune checkpoint blockade antibodies include, but are not limited to, anti-PD-1, anti-PD-L1, and anti-CTLA-4 antibodies. In exemplary embodiments, the antibody is an anti-PD-1 antibody.

The term “antibody” refers to immunoglobulin molecules or other molecules which comprise an antigen binding domain. Antibodies include whole antibodies (e.g., IgG, IgA, IgE, IgM, or IgD), monoclonal antibodies, chimeric antibodies, humanized antibodies, and antibody fragments, including single chain variable fragments (ScFv), single domain antibodies, and antigen-binding fragments, genetically engineered antibodies, among others, as long as the characteristic properties (e.g. ability to bind to the protein of interest or variant) are retained.

The term antibody includes “antibody fragments” or “antibody-derived fragments” and “antigen binding fragments” which comprise an antigen binding domain and displays antigen binding function, for example, Fab, Fab′, F(ab′)2, scFv, Fv, dsFv, ds-scFv, Fd, mini bodies, monobodies, and multimers thereof and bispecific antibody fragments. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), (see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Fragments may comprise a heavy chain variable region (VH domain) and light chain variable region (VL). Fragments may comprise one or more of the heavy chain complementarity determining regions (CDRHs) of the antibodies or of the VH domains, and one or more of the light chain complementarity determining regions (CDRLs), or VL domains to form the antigen binding site.

The term “bispecific” means that the binding protein is able to specifically bind to at least two distinct moieties (e.g., antigen binding sites). Typically, a bispecific molecule comprises two different binding sites, each of which is specific for a different moiety (e.g., antigen). A bispecific molecule may be capable of simultaneously binding two moieties, particularly two moieties expressed on two distinct cells (e.g., a tumor cell and a T cell).

The antibodies or antibody fragments can be wholly or partially synthetically produced. Thus, the antibody may be from any appropriate source, for example recombinant sources and/or produced in transgenic animals or transgenic plants. Thus, the antibody molecules can be produced in vitro or in vivo. The antibody or antibody fragment can be made that comprises all or a portion of a heavy chain constant region, such as an IgG1, IgG2, IgG3, IgG4, IgA1, IgA2, IgE, IgM or IgD constant region.

The terms “specifically binds”, or “binding specificity” refer to the ability of the antibody to form one or more noncovalent bonds with an epitope or antigen via the antibody variable domains. Specificity can be characterized by an antibody-antigen affinity, e.g. as characterized by a dissociation constant (KD) of <100 nM, 10 nM, <1 nM, <0.1 nM, <0.01 nM, or <0.001 nanomolar (nM) (e.g. 10−8 M or less, e.g. from 10−8 M to 10−13 M, e.g., from 10−9 M to 10−13 M).

The terms “protein” or “polypeptide” or “peptide” are used interchangeably to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids. A protein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

In a second aspect, provided herein is a polymeric nanoparticle comprising free thiol groups, wherein the nanoparticle is loaded with a therapeutic nucleic acid operably linked to a promoter. The nanoparticle may be any of the nanoparticles described for use in the methods described herein. The nanoparticle may comprise at least one of thiol-functionalized polyethylene glycol (PEG-SH); and polyethyleneimine (PEI), poly(beta-amino ester) (PBAE), or bioreducible PEI (rPEI). The nanoparticle may comprise a PEI-g-PEG-SH graft copolymer and a branched PEI polymer. In exemplary embodiments, the nanoparticle comprises a) a PEI-g-PEG-SH graft copolymer, wherein the PEG-SH polymers are grafted onto a branched PEI polymer; and b) an additional, non-PEGylated branched PEI polymer. The PEG-SH may have a molecular weight of between about 2 and about 5 kDa. In embodiments, the PEG-SH has a molecular weight of about 5 kDa. The PEI in the graft copolymer and the branched PEI polymer may have a molecular weight of between about 2 and about 25 kDA. In embodiments, the PEG-SH has a molecular weight of about 25 kDa. The PEI-g-PEG-SH and branched PEI may be at a molar ratio of about 1:1. The nanoparticle may be loaded with a therapeutic agent, such as a therapeutic nucleic acid or a small molecule therapeutic.

The polymeric nanoparticle may be formulated into a pharmaceutical composition comprising the nanoparticle and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. Examples of compositions appropriate for such therapeutic applications include preparations for parenteral, subcutaneous, transdermal, intradermal, intramuscular, intracoronarial, intramyocardial, intraperitoneal, intravenous or intraarterial (e.g., injectable), or intratracheal administration, such as sterile suspensions, emulsions, and aerosols. In some cases, pharmaceutical compositions appropriate for therapeutic applications may be in admixture with one or more pharmaceutically acceptable excipients, diluents, or carriers such as sterile water, physiological saline, glucose or the like.

The terms “effective amount” or “therapeutically effective amount” refer to an amount sufficient to effect beneficial or desirable biological and/or clinical results. The amount of the pharmaceutical composition that is therapeutically effective may vary depending on the particular pathogen or the condition of the subject. Appropriate dosages may be determined, for example, by extrapolation from cell culture assays, animal studies, or human clinical trials taking into account body weight of the patient, absorption rate, half-life, disease severity and the like. The dosage lies preferably within a range of circulating concentrations that include the ED50 with little or no toxicity. The dosage may vary within this range depending upon the dosage form employed and the route of administration utilized. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of”those certain elements.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The present disclosure will be more fully understood upon consideration of the following non-limiting examples.

### EXAMPLES

### Introduction

Nanoparticles (NPs) encapsulating gene therapies have shown promise in both preclinical and clinical studies by enabling targeted delivery of gene vectors to a range of therapeutic targets¹⁵⁻¹⁹. Leaky vasculature in GBM can be exploited for the passive accumulation of NPs. However, this passive accumulation, termed the EPR effect, is highly heterogeneous within tumors and only allows approximately 2% of the administered dose to reach the tumor²⁰. To bypass the BB/BTBs, therapeutics can be administered via convection enhanced delivery (CED). While this method is used clinically, it is invasive and is associated with surgical risks^{21,22}. Recently, focused ultrasound (FUS) has emerged as a promising, non-invasive approach to enhance NP delivery and involves the intravenous (IV) administration of FDA-approved gas-filled microbubbles (MBs) at the time of FUS treatment²³⁻²⁵. When exposed to ultrasound waves, MBs oscillate, generating mechanical forces that temporarily disrupt vasculature and allow for therapeutics to pass through. Our group and others have demonstrated that FUS significantly improves NP delivery to tumors²⁶⁻²⁸. Beyond enhancing permeability, FUS has been shown to also decrease interstitial fluid pressure and promote convection transport in tumors, further improving NP delivery²⁶. Clinical trials are currently underway to evaluate the safety and efficacy of repeated FUS treatments in patients with Alzheimer's and GBM, demonstrating the translational potential of this approach^{1,29}.

In addition to carrying diverse payloads, NPs can be functionalized with surface-bound targeting ligands to direct them toward receptors and molecules uniquely expressed or overexpressed by target tissues³⁰. For GBM, NPs are often engineered to bind transferrin receptors³¹, neuropilin-^{132,33}, integrin receptors^{34,35}, and the epidermal growth factor receptor³⁶. Another target that has yet to be explored for NP delivery is exofacial thiols, or free thiols present on the cell surface. Exofacial thiols are enriched within tumors due to high metabolic activity, acidic pH, increased redox activity, and dysregulated protein synthesis in the tumor microenvironment (TME)³⁷⁻³⁹. These disrupted processes likely lead to protein misfolding and exposure of free thiols which readily form disulfide bonds with other free thiols. To leverage this unique tumor feature, we have developed polymeric NPs (SH-NPs) decorated with free thiol groups to target and covalently bind to exofacial thiols in the TME, facilitating transfection. While previous studies have utilized thiol groups for polymer crosslinking⁴⁰⁻⁴², disulfide bond cleavage in oxidative environments^{40,43,44}, immune cell stimulation^{45,46}, and conjugation of targeting ligands or nucleic acids^{44,47-49}, this is the first study to explore exofacial targeting for non-viral gene delivery in GBM.

Polymeric NP formulations offer several advantages over viral vectors for gene delivery. While viral vectors exhibit exceptional transduction efficiency, they pose significant challenges, including risks of insertional mutagenesis, immunogenicity, high production costs, and difficulty in redosing patients^{30,50}. In contrast, NPs provide a safer, more cost-effective alternative with greater flexibility in chemical design and scalability⁵¹⁻⁵⁴. We used a PEGylated-polyethyleneimine (PEI) NP vector. While PEI has shown great promise in preclinical and clinical studies, its high positive charge can cause opsonization and cytotoxicity^{55,56}. To address these limitations, we employ PEGylation, a process that coats NPs with a neutrally charged polyethylene glycol (PEG) layer which allows NPs to evade immune recognition, reduces opsonization, and enhances delivery efficiency^{57,58}.

In this study, we aimed to develop a therapeutic platform to target the dysfunctional BB/BTB in tumors. Previously, our group used plasmids electrostatically bound to cationic MBs and low-pressure FUS to sonoselectively transfect healthy brain tissue, a FUS technique used to selectively transfect the endothelium⁵⁴. Here, by delivering SH-NPs with FUS, we achieved highly selective transfection of the tumor endothelium using a non-viral gene delivery vector. This platform presents a promising strategy to target various dysfunctions of the endothelium for GBM therapy. One application explored here was to enhance immune cell recruitment and infiltration into tumor-bearing mice. To achieve this, we used our sonoselective technique to transfect endothelial cells with a plasmid encoding the CXCL9 chemokine and employed flow cytometry to analyze immune cell recruitment.

### Results

### Exploitation of Exofacial Thiols for Tumor Cell Targeting

Studies have indicated that tumor cells exhibit increased exofacial thiols due to metabolic dysfunction and other cellular abnormalities^{38,39}. However, this phenomenon is not often quantitatively assessed in experiments. To address this, we employed an Ellman's reagent assay to measure free thiols on GL261-Luc2 cancer cells, which is the cell line used for our GBM model, in comparison to an in vitro model of the healthy blood-brain barrier (BBB) using the bEnd.3 endothelial cell line. Our analysis revealed an approximately 1.5-fold increase in exofacial thiols on cancer cells as compared to endothelial cells. To further investigate how the tumor microenvironment (TME) influences endothelial cells, we cultured bEnd.3 cells with GL261-Luc2-conditioned media and assessed cells using the Ellman's reagent. This treatment led to a 10-fold increase in exofacial thiols compared to untreated bEnd.3 cells, and a 6-fold increase compared to the cancer cells. These data highlight exofacial thiols as a promising target for nanoparticle delivery to the TME.

Thiol groups interact with other free thiol groups, such as exofacial thiols, to form disulfide bonds⁵⁹. We developed densely PEGylated PEI NPs with surface-bound free thiol groups (SH-NPs) that covalently bind to exofacial thiols on tumor cells. These SH-NPs are approximately 49 nm in size with a slightly positive surface charge of +2 mV (FIG. 1B). Analysis of off-target transfection in mice treated with CED vs IV injections of E2-Crimson reporter plasmid-loaded SH-NPs showed minimal off-target transfection in all major organs (heart, liver, spleen. And kidneys) for both administration techniques. While no off-target transfection was detected following CED in the lungs, some transfection was observed after IV injections (FIG. 1C).

### Characterization of SH-NP Cellular Transfection Tropism

We used flow cytometry to profile the cellular tropism (i.e., preference) of transfection across different cell types in the TME after administering SH-NPs loaded with an H-2K^{k} reporter plasmid driven by a CMV promoter (CMV-H2-K^{k}). We evaluated tumor cells (CD31⁺), endothelial cells (EphA2⁺), astrocytes (ASCA-2⁺), pericytes (CD146³⁰), and leukocytes (CD45⁺) following IV injection, CED, and FUS+IV. This was done to better understand transfection efficacy per cell to inform therapeutic payload selection for future studies. As expected, IV administration of SH-NPs resulted in poor transfection across all cell types. However, both CED and FUS+IV significantly improved transfection in all cells. While CED enhanced transfection compared to IV alone, FUS+IV resulted in superior transfection in endothelial cells (EC, 73% vs 47%), astrocytes (As, 39% vs 8%), pericytes (Per, 38% vs 13%), and leukocytes (Leu, 42% vs 3%, FIG. 1D-H). Although FUS+IV outperformed CED in these cell types, transfection efficiency of TCs was comparable between the two methods. Furthermore, ECs were the most efficiently transfected, with FUS+IV nearly doubling that of CED, indicating transfection tropism for ECs (FIG. 1E). Examining H2-K mean fluorescence intensity (MFI) revealed that both CED and FUS+IV demonstrated higher MFI than IV alone (FIG. 1I-M). CED achieved the highest MFI in TC, As, and Pe, indicating more H2-K^{k} reporter protein production within these transfected cells. However, FUS+IV resulted in the greatest H2-K^{k} MFI in ECs, and FUS+IV and CED demonstrated comparable H2-K^{k} MFI in the Leu population.

We also aimed to investigate whether the timing of SH-NP delivery relative to FUS treatment would impact cellular tropism or transfection efficacy^{26,60}. The FUS data in FIG. 1D-H (n=9) were obtained from smaller groups of mice treated with SH-NPs either 15 minutes before (−15, n=3), at the time of (0, n=3), or 15 minutes after (+15, n=3) FUS treatment. For all cell types, there was no significant difference in transfection efficacy across the different timing intervals (FIG. 1N-R). Similarly, H2-K^{k} MFI for all tested time points was consistent across cell types (FIG. 1S-W). Extensive characterization of cellular transfection, as shown here, provides valuable insights for selecting appropriate therapeutic payloads and experimental design for the SH-NP platform.

Although PEI is a polymer that is widely used for the encapsulation and delivery of nucleic acids, its strong cationic nature makes it inherently cytotoxic. To mitigate this, SH-NPs are densely PEGylated to reduce toxicity. However, the development of targeted gene delivery platforms formulated with biocompatible polymers could offer a safer alternative. As such, the principle of decorating NPs with free thiols to achieve enhanced tumor targeting can be extended to other NP formulations using biocompatible polymers like poly(beta-amino ester) (PBAE). In this case PBAE can be modified with 5-kDa thiol to create thiol-decorated PBAE NPs. Furthermore, polymeric NP biocompatibility could be further improved by substituting conventional PEI for bioreducible PEI. Bioreducible PEI (rPEI) NPs can be synthesized via a Michael addition reaction as follows. First, PEI hydrochloride linear is dissolved in methanol with N,N-Bis(acryloyl)cystamine, followed by dialysis. Thiolated PEG-PEI (PEI-g-PEG-SH) consisting of 25 kDa PEI, 5 kDa PEG, and a PEG substitution degree of 20%, purchased from Nanosoft can then be used for create PEI-g-PEG-SH/rPEI/pDNA NPs at a nitrogen-to-phosphate (N/P) ratio of 18 using a blend of 25% rPEI and 75% PEI-g-PEG-SH based on amine content.

### Sonoselective Transfection of the Endothelium in Glioblastoma

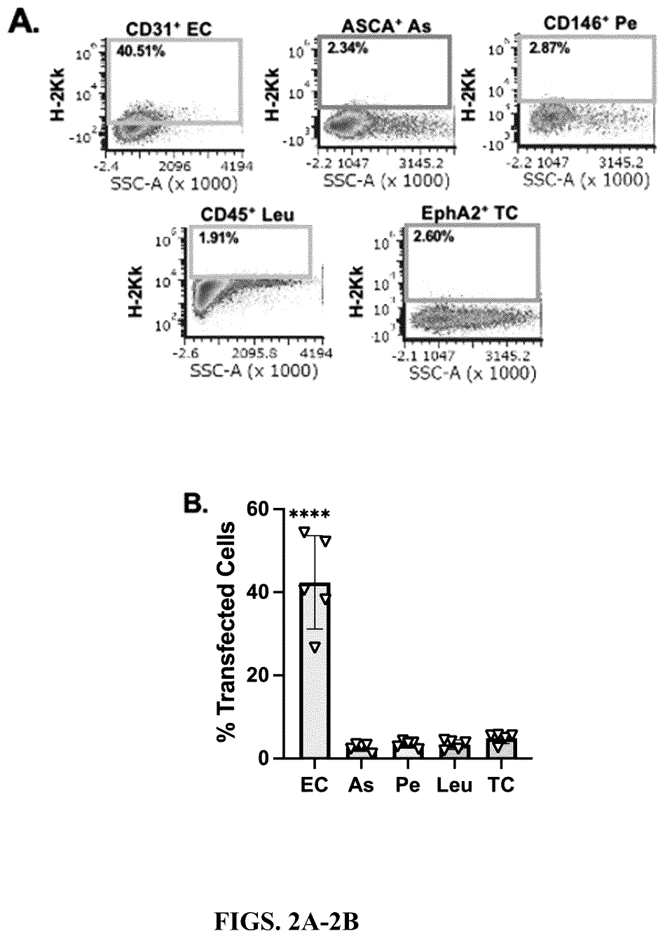

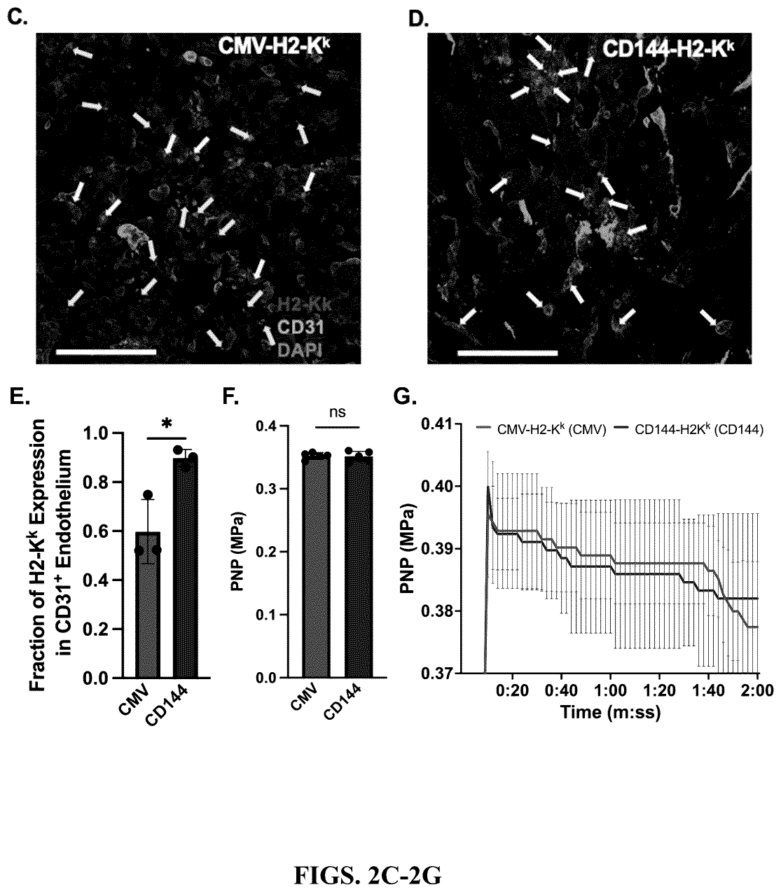

To further enhance the endothelial cell selectivity of this platform, we loaded SH-NPs with H2-K^{k}-encoding plasmids driven by CD144, an endothelial cell-specific promoter (CD144-H2-K^{k}), instead of CMV-H2-K^{k}. Again, we used FUS to deliver SH-NPs and employed flow cytometry to assess cellular transfection (FIG. 2A). By delivering SH-NPs loaded with CD144-H2-K^{k} plasmids via FUS, we successfully achieved sonoselective transfection of the endothelium, with over 40% transfection of endothelial cells and negligible transfection in As, Pe, Leu, and TC (FIG. 2B). Immunofluorescence imaging confirmed that while FUS-mediated delivery of SH-NPs encapsulating CMV-H2-K^{k} transfected both endothelial and non-endothelial cells in the TME (FIG. 2C), transfection following FUS-mediated delivery of SH-NPs loaded with CD144-H2-K^{k} was highly selective for endothelial cells (FIG. 2D), further supported by quantification of H2-K^{k} expression (FIG. 2E). FUS treatments were performed using a clinically relevant FUS technique, passive cavitation detection (PCD), that provides real-time acoustic emissions to monitor and control treatments, ensuring safety^{61,62}. PCD dynamically adjusts the FUS intensity by lowering the treatment pressure when excessive MB activity is detected. Pressure over the course of FUS treatments and average pressure per treatment (0.35 MPa) was comparable between mice that received SH-NPs encapsulating CMV-H2-K^{k} and CD144-H2-K^{k}, indicating similar mechanical energy deposition during treatments (FIG. 2F-G).

### Improved Immune Cell Recruitment Using Sonoselective Transfection of Chemokines

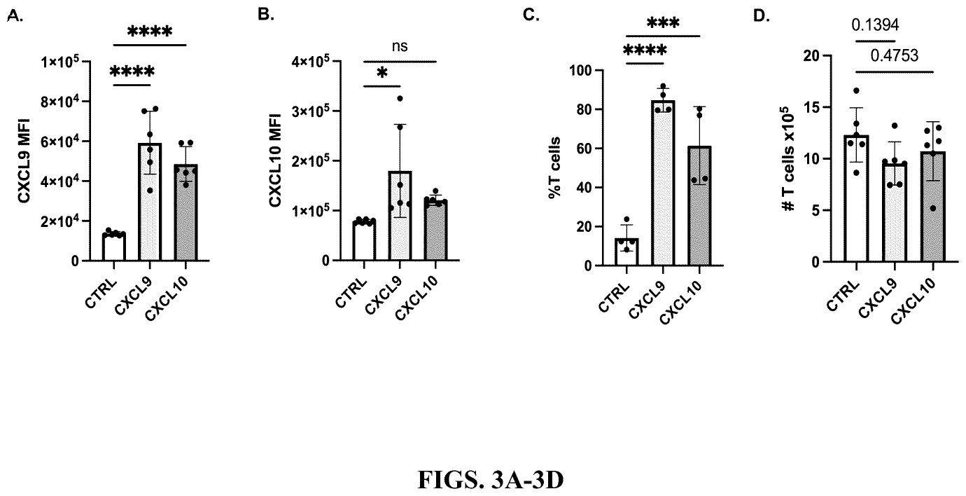

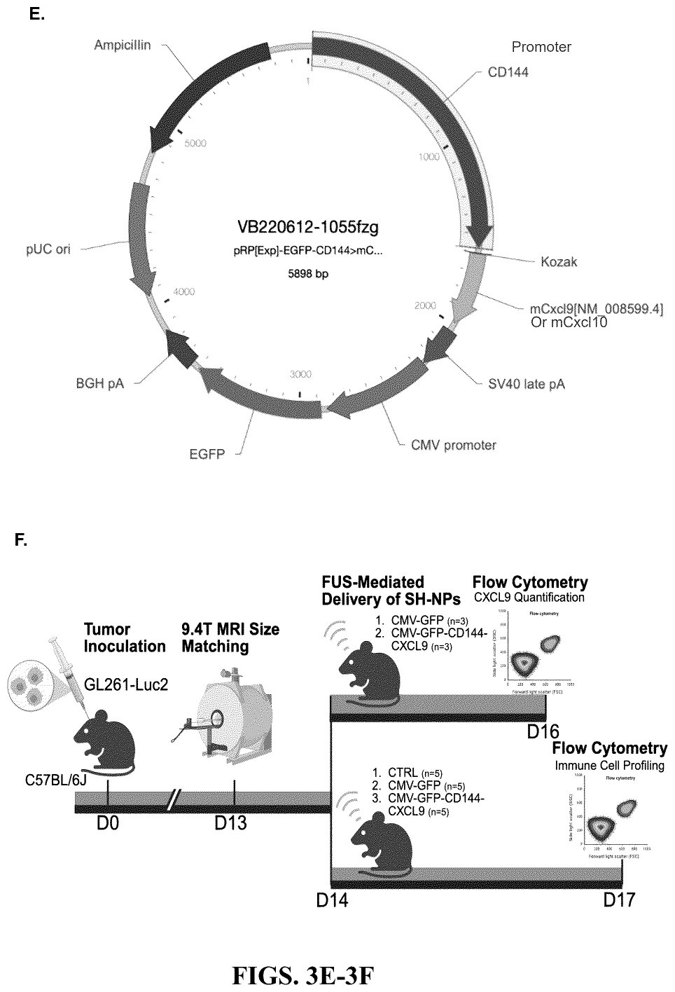

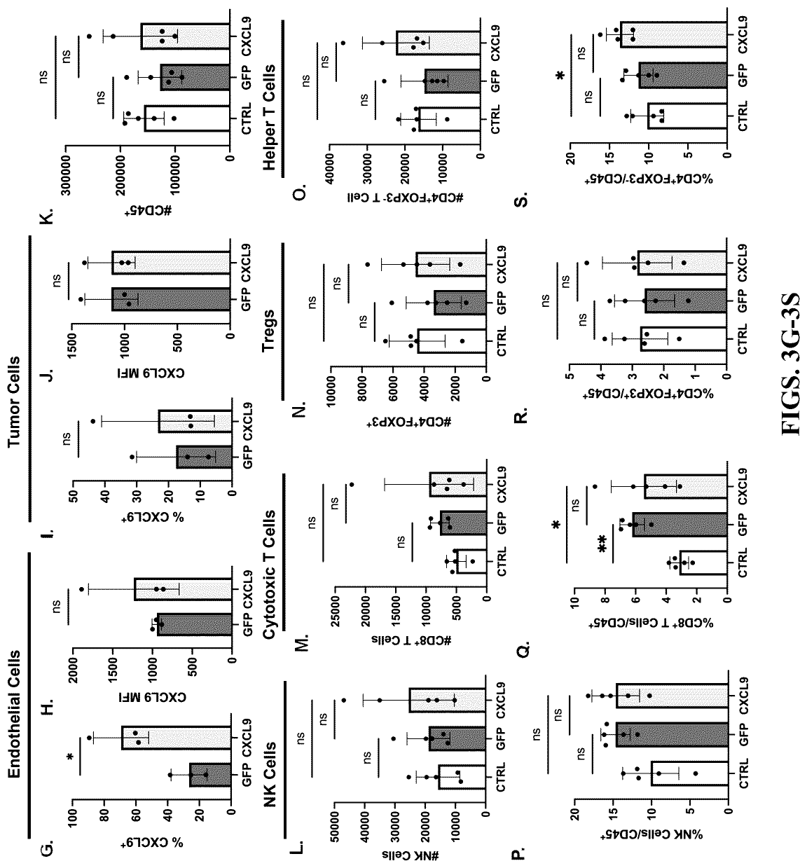

Tumor-induced dysfunctional angiogenesis leads to the formation of abnormal vasculature which secretes pro-tumor factors promoting recruitment of pro-tumor immune cells¹¹, like regulatory T cells (Tregs). Furthermore, tumor vasculature exhibits decreased adhesion molecule expression, further hindering immune cell infiltration in an already immune-privileged organ like the brain¹¹. In this study, we use SH-NPs to target the BTB in an effort to create a more anti-tumor environment. Chemokines, such as CXCL9 and CXCL10, act as signaling molecules that guide immune cells to specific sites in the body^{63,64}. These chemokines are particularly important in the brain for recruiting CXCR3-expressing immune cells, including T cells and NK Cells65. By transfecting endothelial cells in the TME with plasmids encoding these chemokines, we aimed to improve the recruitment, extravasation, and infiltration of anti-tumor immune cells into immunologically cold glioblastomas (FIG. 3D). To test whether CXCL9 levels can be upregulated using gene therapy, we first used an in vitro BBB model using bEnd.3 cells to transfect plasmids encoding CXCL9 or CXCL10 chemokines.

These plasmids were driven by the endothelial-specific CD144 promoter, with a GFP reporter protein driven by the CMV promoter (FIG. 3E). As CXCL9 and CXCL10 are co-regulated, we were interested in understanding how the transfection of one chemokine might influence the expression of the other. Transfection of bEnd.3 cells with the CXCL9-encoding plasmid resulted in a marked increase in the MFI for both CXCL9 and CXCL10 (FIG. 3A-B). Interestingly, when transfecting bEnd.3 cells with the CXCL10-encoding plasmid, CXCL10 MFI increased modestly from 7.8e4 to 1.2e5, while CXCL9 MFI significantly increased to 1.8e5. To determine whether the upregulation of these chemokines could effectively recruit immune cells, we performed a co-culture assay using immortalized cytotoxic T cells and chemokine-transfected bEnd.3 cells. Four days post-transfection, we assessed T cell recruitment. Although the cell counts of T cells remaining in Transwells® was not statistically significant (FIG. 3D), flow cytometry revealed a clear increase in T cell recruitment in both CXCL9-and CXCL10-transfected groups (FIG. 3C). Given that CXCL9 was significantly upregulated following the transfection of either CXCL9 or CXCL10, we chose to examine CXCL9 further in our in vivo experiments.

We next sought to investigate whether sonoselective transfection of endothelial cells within GBM tumors could modulate immune cell recruitment. Following the timeline in FIG. 3F, we first confirmed that in vivo transfection with the CXCL9 plasmid led to a significant increase in the percentage of endothelial cells expressing CXCL9 compared to GFP-transfected controls (FIG. 3G). Interestingly, the MFI of CXCL9 among the CXCL9⁺ endothelial population did not differ (FIG. 3H). As expected, tumor cells showed no difference in the percentage expressing CXCL9 or in CXCL9 MFI.

To assess changes in immune infiltration, we treated GL261-Luc2 tumor-bearing mice in three groups: (1) untreated controls (CTRL), (2) FUS-treated mice receiving SH-NPs encapsulating a CMV-GFP reporter plasmid to control for GFP-reporter immunogenicity (GFP), and (3) FUS-treated mice receiving SH-NPs loaded with CXCL9 plasmids (CXCL9). Four days post-treatment, tumor-bearing brain quadrants were harvested for flow cytometry to assess infiltration of NK cells, cytotoxic T cells, helper T cells, and Tregs. While NK cell and Treg populations remained unchanged, both GFP-and CXCL9-treated mice exhibited a ˜3-fold increase in cytotoxic T cells as compared to the CTRL group. Notably, helper T cells were significantly elevated in the CXCL9-treated group (10.7%) compared to the CTRL (7.4%) and GFP (8.5%) groups. These findings highlight the potential of SH-NPs to target the BTB to ameliorate dysfunction.

Sonoselective Transfection of CXCL9 Improves Survival in Glioma-Bearing Mice Following Treatment with aPD-1

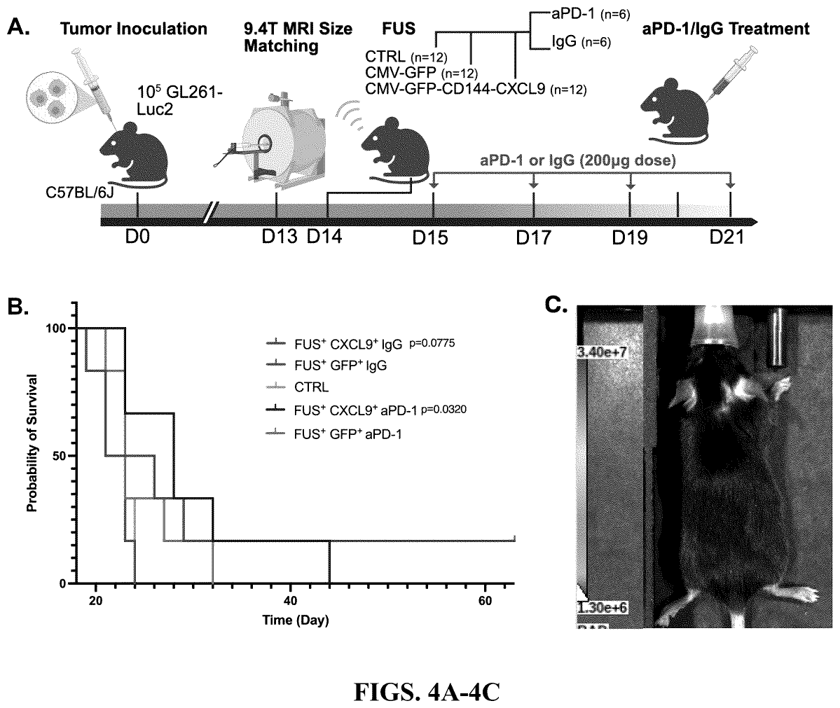

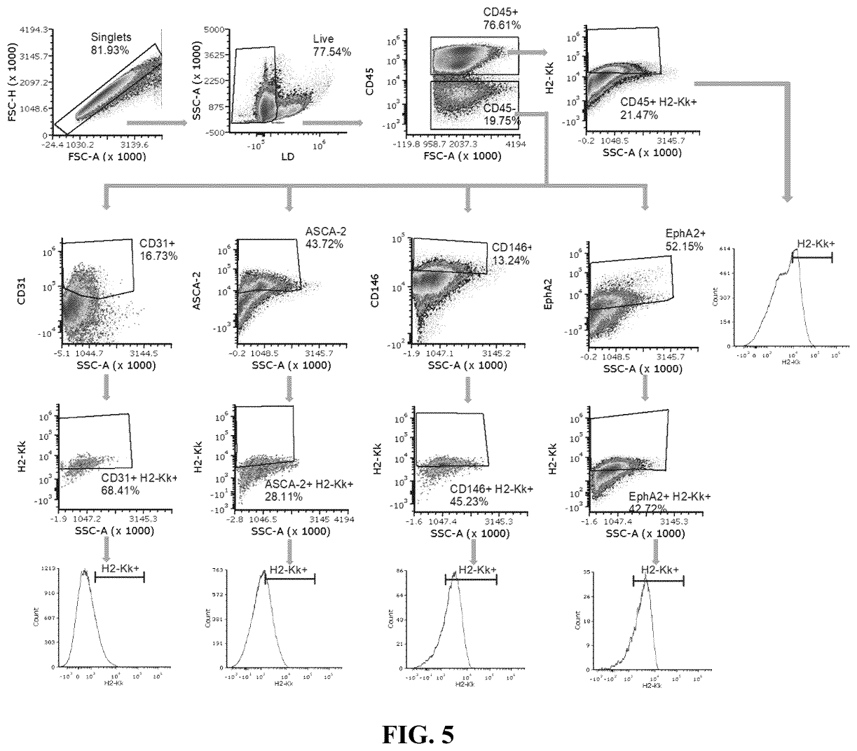

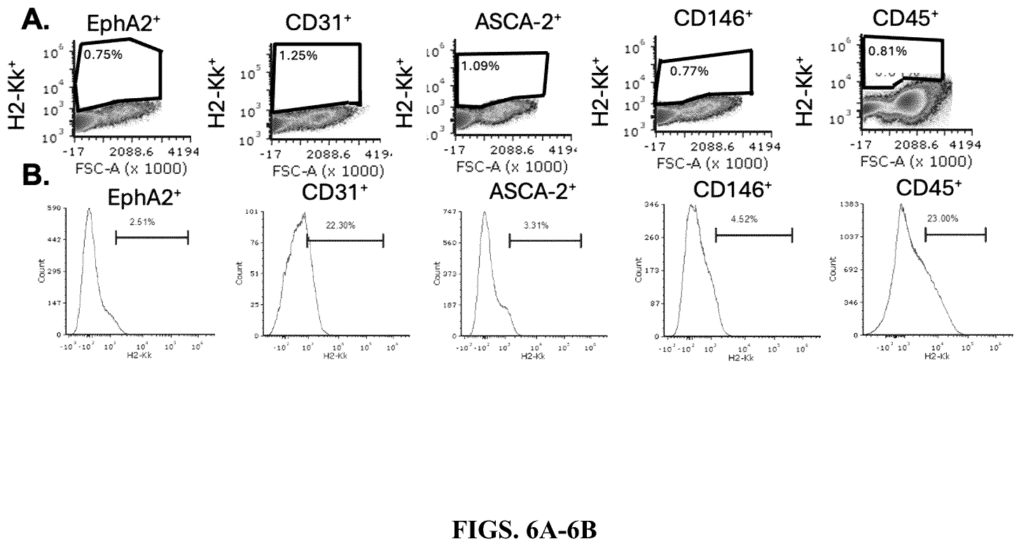

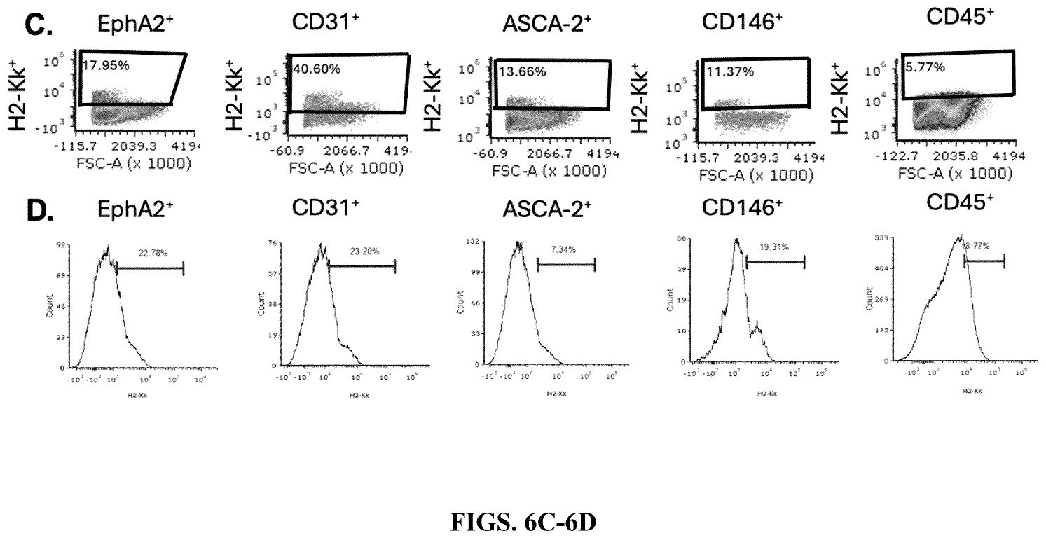

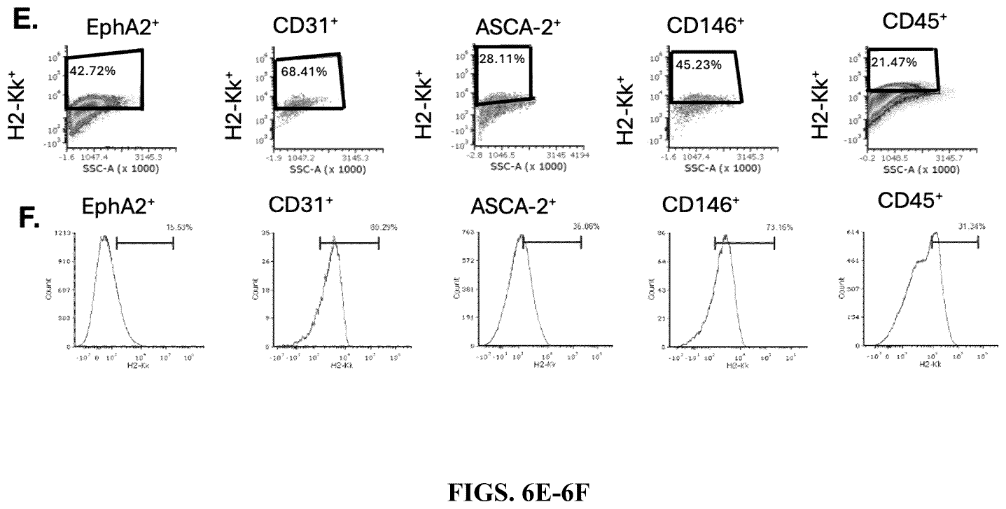

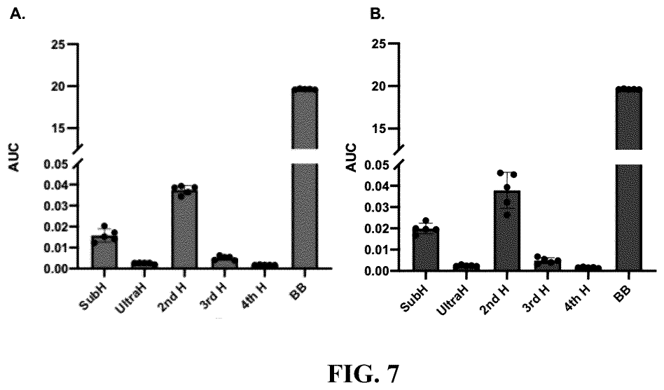

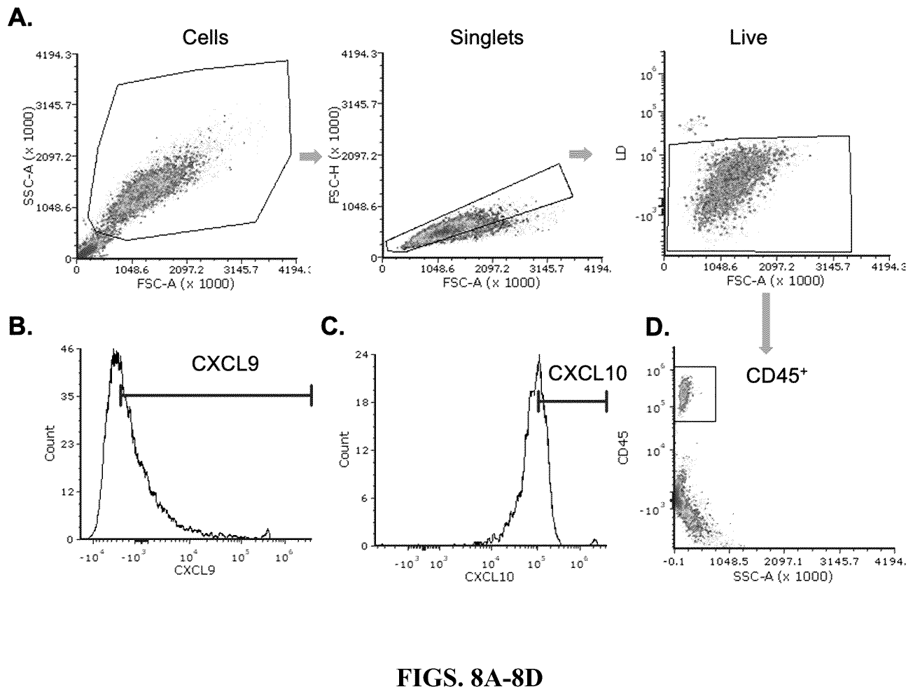

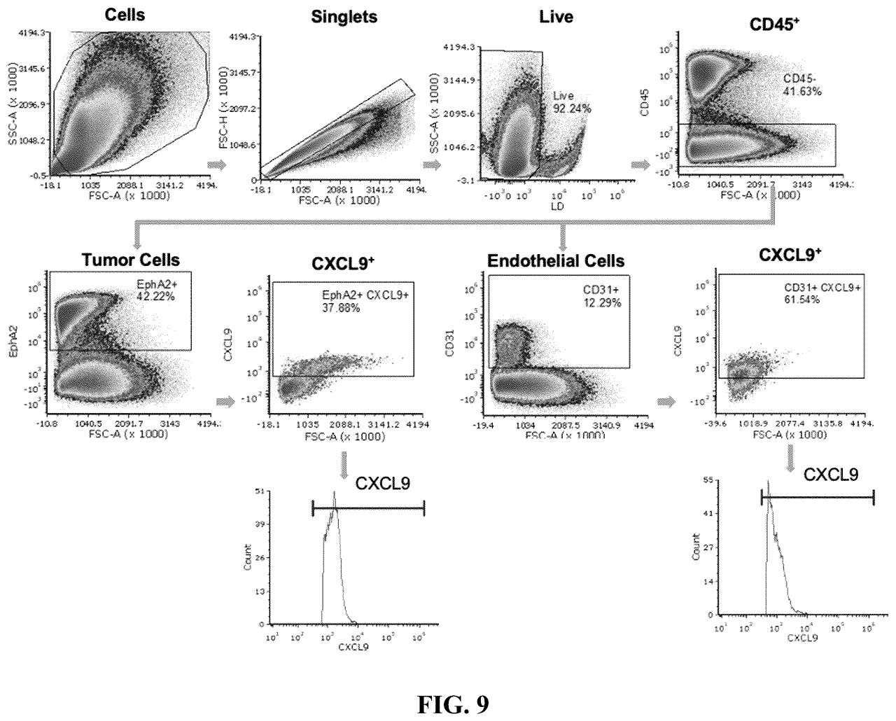

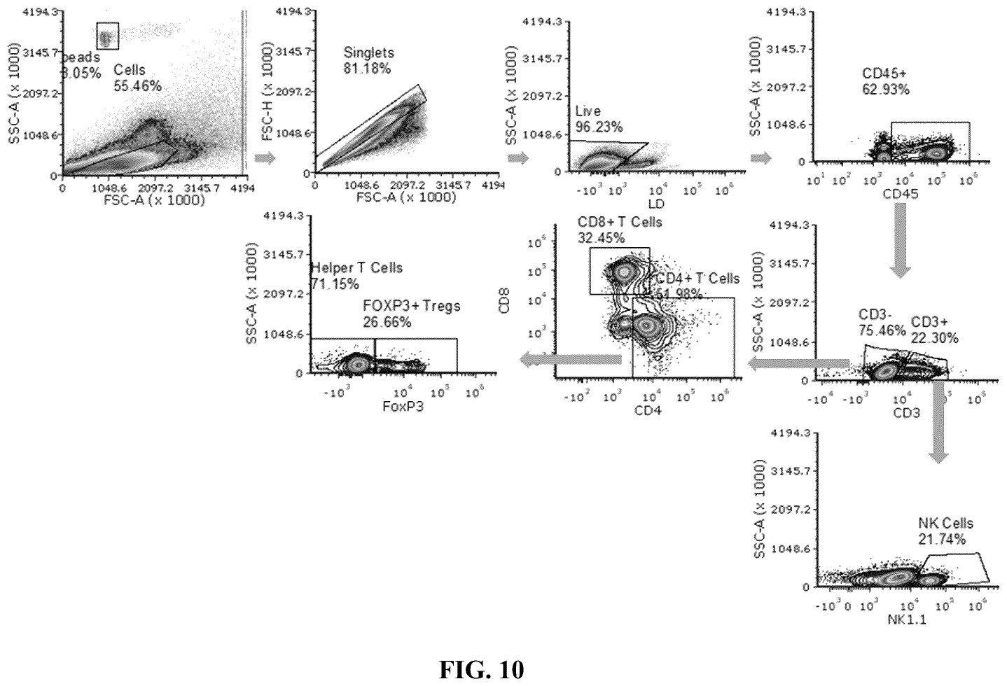

To evaluate whether enhanced recruitment of cytotoxic and helper T cells could augment the efficacy of immune checkpoint blockade, we conducted a survival study. Following tumor implantation, on day 12, FUS was used to deliver either CXCL9 or GFP plasmids. Beginning on day 15, animals received four doses of either aPD-1 or IgG (200 μg) administered every other day (FIG. 4A). One cohort of mice did not receive any additional treatments post tumor implantation to serve as a control. We observed a significant improvement in survival following this combination therapy (FIG. 4B). Notably, CXCL9 plasmid monotherapy also trended toward improved survival, and one mouse in this cohort demonstrated complete tumor eradication (FIG. 4C).

### Discussion

Glioblastoma remains an incredibly difficult cancer to treat with standard of care therapies yielding a median survival of only 15 months. Given this poor prognosis, there is an urgent need for novel therapeutic strategies that enhance treatment efficacy. Selectively transfecting tumor endothelium presents a promising approach to addressing dysfunction caused by rapid angiogenesis, with the potential to restore normal vascular function and improve current therapeutic outcomes. In this study, we use FUS to enhance the delivery of novel thiol-decorated polymeric nanoparticles, SH-NPs, to tumors, achieving highly efficient and selective endothelial cell transfection.

### Development of Targeted SH-NPs

Cells within the TME are enriched with exofacial thiols due to dysregulated protein synthesis, increased metabolic activity, and the acidic pH, all of which are characteristic of tumors^{4, 8, 39, 45, 46}. To exploit this unique pathological hallmark, we have engineered SH-NPs to contain surface-bound thiol groups. Free thiols are highly reactive and form strong covalent bonds with other thiol groups³⁸, like exofacial thiols, which in the context of NP delivery, can facilitate cellular uptake and internalization. SH-NPs also possess favorable physicochemical properties, including a small size and neutral charge, which enable them to pass through dense, highly negatively charged extracellular matrix in brain tissue⁵⁸. Though minimal off-target transfection was observed following systemic administration of SH-NPs in the heart, liver, spleen and kidneys, further investigation into the lungs is necessary to ensure safety.

### Characterization of Cellular Transfection Tropism of SH-NPs

We used flow cytometry to characterize the transfection tropism of SH-NPs across various cell types in the TME to better inform therapeutic payloads for future studies. While in vivo transfection is typically analyzed by immunofluorescence or in/ex vivo fluorescence imaging^{66, 67}, these methods are limited in their ability to quantify transfection efficiency across multiple cell types and lack of cellular resolution, respectively. Flow cytometry offers a powerful alternative that can be used to analyze individual cells, providing a comprehensive understanding of a gene delivery platform and its transfection capabilities. In this study, SH-NPs were loaded with a CMV-H2-K^{k} reporter plasmid, and flow cytometry was used to quantify both the percentage of transfected cells and the MFI of transfected cells, providing insights into both the number of successfully treated cells and the extent of transgene expression per cell type.

NPs for GBM therapy can be administered via several different routes, each with distinct advantages and limitations⁶⁸. As such, we evaluated the efficacy of SH-NPs delivered via IV, CED, and IV+FUS. As expected, IV administration alone resulted in poor transfection due to its reliance on the enhanced permeability and retention (EPR) effect, which is inefficient for NP delivery, even for targeted formulations like SH-NPs^{7,8}. While CED improved transfection, FUS+IV significantly outperformed CED in all cell types other than tumor cells, suggesting a synergistic effect between SH-NPs and FUS. This is likely due to FUS-mediated upregulation of cellular uptake mechanisms, such as transcytosis and endocytosis, as well as increased vascular permeability from tight junction disruption⁶¹. Additionally, some studies demonstrate that higher-pressure FUS can modulate the presence of free thiols⁶⁹⁻⁷¹. Further investigation is needed to determine whether lower-pressure FUS applied with MBs, as used here, could induce a similar effect, thereby exposing more exofacial thiols for SH-NP targeting. The similar transfection efficacy of tumor cells following CED and FUS+IV is likely due to differences in access to tumor cells per delivery modality. CED offers direct injection of therapeutics deep into the tumor core where vasculature is often sparse or poorly perfused, whereas SH-NPs administered via IV must pass through the vasculature to reach tumor cells, limiting their accumulation in deeper tumor regions. Additionally, MB oscillation is confined to perfused tumor regions, so deeper, non-perfused tumor regions would not experience FUS-induced mechanical stimulation of transport mechanisms.

We also evaluated whether the timing of SH-NP administration relative to FUS influenced transfection efficacy as optimal timing for NP delivery relative to FUS treatment remains poorly characterized and is likely dependent on individual NP formulations^{26,60}. SH-NPs were administered either 15 minutes before, at the time of, or 15 minutes after FUS treatment. No significant differences in transfection efficiency or MFI were observed within this 30-minute timeframe. This extensive characterization of SH-NP transfection provides valuable insights for optimizing experimental timelines and informing the design of future therapeutic strategies.

A particularly noteworthy observation is that approximately 73% of endothelial cells were successfully transfected. Moreover, endothelial cells transfected via FUS+IV exhibited the highest MFI, indicating superior transgene expression per cell. While non-viral delivery vectors are generally less efficient than viral vectors, this approach achieves transfection efficiencies comparable to adeno-associated viruses (AAVs)^{72,73}, highlighting its potential as a non-viral gene delivery strategy. Additionally, despite their nature to phagocytose foreign material, about 43% of leukocytes (CD45⁺ cells) were transfected following FUS+IV. Immune cells within the TME are exposed to oxidative stress that may increase presence of exofacial thiols and facilitate immune cell transfection. Further investigation into tropism for specific CD45⁺ immune subtypes can inform therapeutic payloads for potential immunomodulatory therapies for GBM. These data also demonstrate the versatility of therapeutic platforms for FUS-mediated delivery of SH-NPs to screen different therapies for GBM.

### Sonoselective Transfection of the Tumor Endothelium

To further improve tropism for endothelial cells, we loaded SH-NPs with an H2-K^{k} reporter plasmid driven by CD144, an endothelial-specific promoter. Using flow cytometry, we profiled transfection across cells within the TME and found efficient transfection specifically in the endothelium, with negligible transfection of other cell types. Notably, when using the CD144-H2-K^{k} reporter plasmid, we observed transfection in 42% of endothelial cells, compared to 73% using the CMV-H2-K^{k} plasmid. As energy FUS energy deposition MB activity were comparable between treatments, this difference is likely attributed to the use of the CD144 promoter, which is not as efficient as the CMV promoter in driving gene expression. Nevertheless, by leveraging FUS to deliver CD144-H2-K^{k}-loaded SH-NPs, we achieved highly selective transfection, or sonoselective transfection, of endothelial cells, demonstrating the potential of this platform to precisely and efficiently target the dysregulated endothelium within the TME.

### Using Sonoselective Transfection to Improve Immune Cell Recruitment in GBM

We next sought to explore the therapeutic potential of this platform for GBM. In tumors, endothelial cells exhibit reduced adhesion molecule expression and secrete factors that create an immunosuppressive environment, limiting the infiltration of anti-tumor immune cells¹¹. Importantly, CXCL9 and CXCL10 chemokines recruit CXCR3-expressing immune cells, like T cells, NK cells, macrophages, and microglia⁶⁵. While some studies suggest that these chemokines drive an anti-tumor response, others indicate a pro-tumor effect, likely dependent on the specific immune cell populations recruited⁶⁴. We aimed to investigate whether sonoselective transfection of the endothelium using SH-NPs could modulate immune infiltration and elicit an anti-tumor response.

First, we used an in vitro model to confirm that increased chemokine expression could enhance the recruitment of anti-tumor immune cells. Following bEnd.3 transfection with CXCL9 and CXCL10 plasmids, flow cytometry analysis of a co-culture assay revealed that while both chemokines facilitated cytotoxic T cell recruitment, CXCL9 elicited a more robust response. As such, we selected CXCL9 for in vivo studies.

Following sonoselective transfection of CXCL9-loaded SH-NPs, we used flow cytometry to assess immune cell infiltration within the TME. Notably, the population of Tregs, known for their pro-tumorigenic activity, remained unchanged, demonstrating that transfection of endothelial cells with CXCL9 does not promote pro-tumor immune cell recruitment. Interestingly, we observed an increase in cytotoxic T cell populations following treatment with both GFP and CXCL9 plasmids. We hypothesize that the SH-NP formulation itself may modulate the immunosuppressive TME by altering redox dynamics within the TME. Reactive oxygen species (ROS), which are elevated in tumors, can impair the activity of NK and T cells, contributing to immune suppression. In response to elevated ROS levels, tumors upregulate glutathione, a natural antioxidant that neutralizes ROS via its free thiol group. The surface of SH-NPs is rich in free thiols and as such, may mimic this antioxidant function by reducing ROS levels and thereby restoring immune function. While this hypothesis requires further investigation, it may explain the enhanced recruitment of cytotoxic T cells observed with both treatment groups. Nevertheless, CXCL9 transfection led to a significant increase in helper T cell recruitment, which could support immune licensing and recruitment of additional immune cells to the tumor. Further analysis at extended timepoints will be critical for elucidating the full impact of CXCL9-mediated immune modulation.

Although sonoselective transfection of CXCL9 increased cytotoxic and helper T cell infiltration into the TME, it is likely that immunosuppressive signals continued to dampen T cell activity and tumor recognition. To overcome this limitation, we investigated the addition of aPD-1 immune checkpoint blockade with CXCL9 transfection. This combination significantly improved survival, highlighting its translational potential given the established clinical use of aPD-1 therapies. Interestingly, one mouse in the FUS⁺CXCL9⁺ IgG group achieved complete tumor eradication, suggesting that CXCL9-driven immunomodulation alone may be sufficient to drive an anti-tumor response in some cases. Further characterization of TME changes after sonoselective transfection will be required to elucidate the mechanisms underlying this effect.

Overall, these results demonstrate that sonoselective transfection of CXCL9 using a non-viral vector can improve survival in GBM models. Future studies exploring the optimal timing of aPD-1 administration relative to CXCL9 delivery, as well as integration with additional therapeutics such as chemotherapy, may further enhance therapeutic efficacy.

### Conclusion

In this study, we developed a novel therapeutic platform that leverages FUS to selectively and efficiently transfect endothelial cells within the TME. As tumors progress, dysfunction of the endothelium drives several dysregulated processes that promote tumor progression and hinder therapeutic efficacy. By achieving highly specific endothelial cell targeting, this platform enables screening of a broad range of therapeutic targets within the endothelium. Using this platform, treatment of mice with a combination therapy of sonoselective transfection of tumor endothelium with CXCL9 and aPD-1 demonstrated improved survival, indicating the potential for future clinical translation. Furthermore, beyond the approach explored in this study, given the rapid angiogenesis in tumors, anti-angiogenic gene therapies could be delivered to inhibit aberrant vascular growth. Furthermore, CRISPR-based gene editing could be leveraged to suppress efflux pump activity in the endothelium, enhancing chemotherapy retention and efficacy. Moreover, as exofacial thiols are not unique to GBM, this platform holds promise for translation to other solid tumors, providing a versatile tool for gene therapy screening across multiple cancer types. Ultimately, this platform offers a powerful and adaptable strategy to investigate and optimize therapeutic interventions targeting the tumor endothelium.

### Methods

### In Vitro Free Thiol Quantification

Exofacial thiols were quantified in the bEnd.3 and GL261-Luc2 cell lines using the Ellman's reagent as described the manufacturer - a colorimetric assay used to quantify free thiol groups. GL261-Luc2 cells were maintained in high glucose DMEM (Gibco) supplemented with 10% FBS, 1 mM sodium pyruvate, 1 mM non-essential amino acids (Gibco), and 100 ug/mL geneticin (Gibco). bEnd.3 cells were maintained in high glucose DMEM (Gibco) supplemented with 10% FBS (Gibco) and 1 mM sodium pyruvate (Gibco). For media stimulated cells, GL261-Luc2 cells were cultures with complete media. Following 2 days, media was harvested, centrifuged at 1200 RPM to remove debris, plated onto bEnd.3 cells, and cultured for 2 additional days. Ellman's reagent (GoldBio) was used to quantify free thiol groups as described by the manufacturer (ThermoFIsher Scientific). Briefly, once cells reached 70-80% confluency, 5*10⁵ cells were harvested and resuspended in 250 uL of PBS and incubated with 2.5 mL reaction buffer and 50 uL Ellman's reagent for 15 min. A spectrophotometer was then used to measure sample absorbance at 412 nm.

### In Vitro Transfection

bEnd.3 cells were maintained as described above and seeded in 6 well plates at 1*10⁵ cells per well and transfected with CXCL9 (VB220612-1055fzg, Vectorbuilder) or CXCL10 (VB220426-1203dpz, Vectorbuilder) plasmids using Lipofectamine3000 according to the manufacturer (ThermoFisher). Cells were analyzed 4 days later using flow cytometry to assess chemokine expression. 24 hours and 12 hours prior to analysis, cells were treated with 500 U/mL recombinant mouse IFN-γ (R&D Systems) and 1×brefeldin-A (ThermoFisher), respectively. Cells were harvested and stained with Live/Dead Fixable Blue dye (1:1000, ThermoFisher) and then incubated with FcBlock. Following methanol permeabilization, cells were stained with aCXCL9 (1:200, Biolegend, 515606), CXCL10 primary antibody (1:200, ThermoFisher, 701225), and aCD45 (1:100, ThermoFisher, A15395). Cells were then washed and stained with CXCL10 secondary (1:1600, Goat anti-Rabbit AF555, ThermoFisher, A-21428). Data was acquired using the 5-laser Aurora Borealis spectral cytometer (Cytek) and analysis was performed using the FCS Express software (De Novo Software).

### T Cell Co-Culture Assay

Immortalized cytotoxic T cells (CTLL-2, ATCC) were maintained in RPMI-1640 Medium (Gibco) supplemented with 2 mM L-glutamine (Gibco), 1 mM sodium pyruvate (Gibco), 10% FBS (Gibco) and 10% T-STIM with Con A (Becton Dickinson). Four days after bEnd.3 transfection, as described above, cells were moved into wells of a 24 mm Transwell® (Corning). CTLL-2 cells were pipetted into the 3.0 μm pore polyester membrane inserts at a concentration of 1*10⁵ cells per insert. Plates were incubated for 90 min at 37° C. CTLL-2 cells in Transwell® inserts were counted using an automatic cell counter (Luna). Media containing recruited CTLL-2 cells and bEnd.3 cells were harvested and processed for flow cytometry. First, cells were stained with Live/Dead Fixable Blue dye (1:1000, ThermoFisher) and then incubated with FcBlock. Next, cells were stained with aCD31 (1:100, ThermoFisher, RM5228) and aCD45 (1:100, ThermoFisher, A15395). Data was acquired using the 5-laser Aurora Borealis spectral cytometer (Cytek) and analysis was performed using the FCS Express software (De Novo Software).

### SH-NP Formulation and Characterization

Polymers used for SH-NP formulation are (1) PEI-g-PEG-SH graft copolymer of 25 kDa branched PEI modified with a 20% substitution ratio of 5 kDa SH-PEG (Nanosoft Polymers) and (2) 25 kDa branched PEI (ThermoFisher). Polymers solutions were prepared at a PEI-g-PEG-SH:PEI molar ratio of 3. The polymer solution is incubated with tris(2-carboxyethyl)phosphine (TCEP) and a 10:1 ratio for 10 min to reduce disulfide bonds within the polymer network. SH-NPs were then formed by the drop-wise addition of 10 volumes of pDNA diluted in water to 1 volume of polymer solution (while vortexing) at an optimized nitrogen to phosphate (N/P) ratio of 6. The SH-NP solution is then purged with decafluorobutane (DFB) gas to remove oxygen and washed once using Amicon® Ultra Centrifugal Filters (100,000 MWCO, Sigma Aldrich) filled with DFB gas to prevent reformation of disulfide bonds and remove free polymer and TCEP. SH-NPs were concentrated to 1 mg/mL and stored in inert gas and used same day. SH-NPs were loaded with a CMV-H2-K^{k} plasmid (130-092-083, Miltenyi) or CD144-H2-K^{k} plasmid (VB240821-1392jvd, VectorBuilder) for flow cytometry transfection characterization studies and with E2-Crimson plasmids (Takara Bio) for ex vivo fluorescence imaging. The hydrodynamic diameter and polydispersity index of SH-NPs were measured using dynamic light scattering and ζ-potential was measured using laser Doppler anemometry using a Zetasizer Nano ZS90 (Malvern Instruments).

### GL261-Luc-2 Intracranial Inoculations

Animal experiments were approved by the University of Virginia Animal Care and Use Committee and conformed to the National Institutes of Health guidelines for the use of animals in research. C57BL/6J mice (Jackson Laboratory) were housed under standard laboratory conditions (22° C. and 12 h/12 h light/dark cycle) and treated between 8 and 10 weeks of age. Cell were resuspended (1×10⁵ cells per 2 μL) in sterile PBS and implanted into the right striatum of mice placed on a stereotactic frame using a syringe pump at a controlled rate of 0.5 μL/min. Cells were injected 2.0 mm lateral from the sagittal suture, 0.5 mm anterior of bregma and 3 mm below the dura.

### Tumor Size Matching

Prior to treatment, tumor volumes were acquired for size matching. MR images of mouse brains were obtained using a 9.4T Bruker MRI. Mice were anesthetized and were retro-orbitally injected with a gadolinium contrast agent (Multihance) at a dose of 0.01 nmol diluted in saline (0.2 uM) prior to T1-weighted image acquisition. Tumor volumes were measured using a DICOM viewer (Horos Project).

### CED of SH-NPs

Fourteen days after inoculation, size matched tumors were treated with SH-NPs. Anesthetized mice were placed on a stereotactic frame. Using the pre-existing burr hole as a guide, a dose of 20 ug of SH-NP solution was injected directly into the tumor 3 mm below the dura using a syringe micropump at a controlled rate of 0.5 uL/min. To analyze cellular tropism, mice were perfused with ice-cold 1×PBS and tumor bearing brain quadrants were harvested 3 days after treatment and processed for flow cytometry. For off-target transfection analysis, mice were similarly perfused with ice-cold 1×PBS and major organs were harvested for LagoX imaging (Spectral).

### FUS-Mediated Delivery of SH-NPs

Mice were anesthetized on day 14 and treated with FUS on size matched tumors to deliver SH-NPs administered via tail vein. Mouse heads were shaved and depilated prior to placement on the stereotactic frame of the RK-50 FUS system (FUS Instruments) and coupled to a degassed water bath with ultrasound gel. Treatments were performed using a 1.1 MHz single element transducer with 3 sonication points per tumor (burst length: 10 ms, burst period: 2000 ms, number of sonications: 60, total sonication time: 2 min). Real time passive cavitation detection (PCD) monitoring was used to modulate sonication pressures. Parameters for this system included a starting pressure of 0.2 MPa, a maximum pressure of 0.4 MPa, pressure increments of 0.05 MPa, pressure drop of 0.95, 20 baseline sonications without microbubbles, AUC bandwidth of 500 Hz, AUC standard deviations of 10, and frequency selection of the first and second ultra-harmonic and the subharmonic. Control mice were injected intravenously with SH-NPs at a dose of 40 μg pDNA (IV). For FUS treatments, mice were injected intravenously with albumin-shelled microbubbles (OPTISON) at a dose of 3*10⁵ microbubbles/g immediately at the start of FUS and SH-NPs were administered at a dose of 40 μg pDNA either 15 minutes before (−15) at the time of (0) or 15 minutes after (+15) the start of FUS treatment (FUS+IV). To analyze cellular tropism, mice were perfused with ice-cold 1×PBS 3 days later and tumor bearing brain quadrants were harvested and processed for flow cytometry and major organs were harvested for LagoX imaging.

### PCD Analysis

Acoustic emissions data were collected with a hydrophone built into the center of the FUS transducer and data was processed using a custom MATLAB script. The area under the curve of the acoustic emissions was calculated at the subharmonic and ultra-harmonics after applying a 300 Hz bandwidth filter. Broadband emissions were calculated by summing acoustic emissions and removing emissions at the fundamental frequency, harmonics, subharmonics and ultra-harmonics.

### Flow Cytometric Analysis of Transfection Tropism

Tumor bearing brain quadrants were processed into single cell suspensions using the Adult Brain Dissociation Kit according as directed by the manufacturer (Miltenyi). Cells were then stained for viability using the Live/Dead Fixable Blue dye (1:1000, ThermoFisher). Cells were then incubated with FcBlock and stained with aCD31 (1:100, ThermoFisher, RM5228), aCD45 (1:100, ThermoFisher, A15395), aEphA2 (1:200, ThermoFisher, MA5-40937), ASCA-2 (Miltenyi, 130-116-243) aCD146 (1:50, ThermoFisher, 25-1469-42), and aH2-K^{k} (1:10, Miltenyi, 130-102-346). Data was acquired using the 5-laser Aurora Borealis spectral cytometer (Cytek) and analysis was performed using the FCS Express software (De Novo Software).

### Immunofluorescent Imaging

Brain samples were embedded in OCT (Fisher 23-730-571), and frozen on dry ice. Frozen blocks were stored at −80° C. before sectioning and staining. Cryo-sections were cut at 20 um thick. The mounted sections were incubated with blocking solution (1% NGS in 2% BSA and 0.1% Tween 20 PBS) for 1 h at room temperature. Sections were next incubated overnight at 4° C. with Alexa Fluor 594 anti-mouse CD31 (1:200, BioLegend, 102520) in antibody solution (2% BSA and 0.1% Tween 20 in PBS). After washing 3× for 10 min in 0.1% Tween 20 PBS, sections were incubated for 20 mins. at room temp with DAPI (1:1000, ThermoFisher, 62248). After final washes in PBS, sections were sealed with ProLong Gold antifade reagent (ThermoFisher, P36930) and cover slipped with cover glass for confocal imaging using the Stellaris confocal microscope (Leica Microsystems).

### Sonoselective Chemokine Transfection

Fourteen days after inoculation, mice were separated into 4 groups and treated accordingly. FUS⁻: no FUS, FUS⁺: FUS treatment of tumors, FUS⁺ GFP: SH-NPs encapsulating CMV-GFP reporter plasmid delivered via FUS, FUS⁺ CXCL9: FUS-mediated delivery of SH-NPs encapsulating CD144-CXCL9 plasmid with CMV-GFP reporter. FUS treatment was applied and tumor-bearing brain quadrants were harvested and processed for flow cytometry. For quantification of CXCL9 expression, tumor-bearing brain quadrants were processed using the Adult Brain Dissociation Kit (Miltenyi). Cells were stained with Live/Dead Fixable Blue dye (1:1000, ThermoFisher) and then incubated with FcBlock. Surface markers were stained using aCD45 (1:100, ThermoFisher, A15395), aCD31(1:100, ThermoFisher, RM5228), aEphA2(1:200, ThermoFisher, MA5-40937), and aCXCL9 (1:200, BioLegend, 515606). For immune cell profiling, tumor-bearing brain quadrants were harvested, minced, and incubated in 1 mg/mL collagenase and 1 mg/mL DNAse for 45 min at 37° C. Myelin was then removed using a Percoll® gradient and the cell suspension was incubated with red blood cell lysis. Cells were stained with Live/Dead Fixable Blue dye (1:1000, ThermoFisher) and then incubated with FcBlock. Surface markers were stained using aCD45 (1:100, ThermoFisher, A15395), aCD4 (1:400, ThermoFisher, M001T02B06), aCD8 (1:200, ThermoFisher, 48-0081-82), aCD3 (1:100, BD Biosciences, 4157782), aNK1.1 (1:200, BioLegend, 108745), aCD11b (1:200, BD Biosciences, 566416), aCD11c (1:200, ThermoFisher, 69-0114-82), and aMHCII (1:500, BD Biosciences, 750281). Cells were fixed and permeabilized using the Foxp3/Transcription Factor Staining Buffer Set (ThermoFisher) and stained with aFoxP3 (1:200, ThermoFisher, 606-5773-82). Data was acquired using the 5-laser Aurora Borealis spectral cytometer (Cytek) and analysis was performed using the FCS Express software (De Novo Software).

aPD-1 and CXCL9 Combination Strategy

Fourteen days after inoculation, mice were separated into 5 groups and treated accordingly. FUS⁺ CXCL9⁺ aPD-1, FUS⁺ CXCL9⁺ IgG, FUS⁺ GFP⁺ aPD-1, FUS⁺ GFP⁺ IgG, and CTRL (receiving no additional treatments after tumor implantation). Mice assigned to antibody treatment received four doses of either aPD-1 or IgG (200 μg per dose) administered intraperitoneally every other day starting on day 15. Mice were weighed every day starting on day 14 and monitored until euthanasia.

### Bioluminescence Imaging of Tumors

To identify tumor presence throughout the survival study, bioluminescence of GL261-Luc2 tumors was imaged. Mice were first injected with 150 mg/kg D-Luciferin (GoldBio) solution intraperitoneally. 10 minutes following the injection, mice were anesthetized and in vivo images were taken using a LagoX (Spectral Instruments).

### Statistical Analysis

All data are reported as mean±standard error of the mean (SEM). The “n” values per group are made evident by individual data points or by text in figure captions. Statistical significance was assessed at p<0.05 for all experiments and details of statistical testing are explained in the figure legends (GraphPad Prism 10).

### REFERENCES CITED

1. Fernandes, C. et al. Current Standards of Care in Glioblastoma Therapy. Glioblastoma 197-241 (2017) doi:10.15586/CODON.GLIOBLASTOMA.2017.CH11.
2. Rong, L., Li, N. & Zhang, Z. Emerging therapies for glioblastoma: current state and future directions. J Exp Clin Cancer Res 41, 142 (2022).
3. Omuro, A. & DeAngelis, L. M. Glioblastoma and other malignant gliomas: a clinical review. JAMA 310, 1842-1850 (2013).
4. Van Solinge, T. S., Nieland, L., Chiocca, E. A. & Broekman, M. L. D. Advances in local therapy for glioblastoma-taking the fight to the tumour HHS Public Access. Nat Rev Neurol 18, 221-236 (2022).
5. Pilotto Heming, C. et al. P-glycoprotein and cancer: what do we currently know? Heliyon 8, e11171 (2022).
6. Wu, D. et al. The blood-brain barrier: Structure, regulation and drug delivery. Signal Transduction and Targeted Therapy 2023 8:1 8, 1-27 (2023).
7. Wu, J. The Enhanced Permeability and Retention (EPR) Effect: The Significance of the Concept and Methods to Enhance Its Application. J Pers Med 11, (2021).
8. Prabhakar, U. et al. Challenges and key considerations of the enhanced permeability and retention (EPR) effect for nanomedicine drug delivery in oncology. Cancer Res 73, 2412 (2013).
9. Callaghan, R., Luk, F. & Bebawy, M. Inhibition of the Multidrug Resistance P-Glycoprotein: Time for a Change of Strategy? Drug Metabolism and Disposition 42, 623 (2014).
10. Waghray, D. & Zhang, Q. Inhibit or Evade Multidrug Resistance P-Glycoprotein in Cancer Treatment. J Med Chem 61, 5108-5121 (2018).
11. Lin, H. et al. Understanding the immunosuppressive microenvironment of glioma: mechanistic insights and clinical perspectives. Journal of Hematology & Oncology 2024 17:1 17, 1-81 (2024).
12. Luciano, R. et al. New Perspectives in Glioblastoma: Nanoparticles-based Approaches. Curr Cancer Drug Targets 17, 203-220 (2016).
13. Játiva, P. & Ceña, V. Use of nanoparticles for glioblastoma treatment: a new approach. Nanomedicine (Lond) 12, 2533-2554 (2017).
14. Alexis, F., Pridgen, E., Molnar, L. K. & Farokhzad, O. C. Factors affecting the clearance and biodistribution of polymeric nanoparticles. Mol Pharm 5, 505-515 (2008).
15. Thomas, T. J., Tajmir-Riahi, H. A. & Pillai, C. K. S. Biodegradable Polymers for Gene Delivery. Molecules 2019, Vol. 24, Page 3744 24, 3744 (2019).
16. Kumar, R. et al. Polymeric Delivery of Therapeutic Nucleic Acids.
17. Wang, X., Niu, D., Hu, C. & Li, P. Polyethyleneimine-Based Nanocarriers for Gene Delivery. Curr Pharm Des 21, 6140-6156 (2015).
18. Sayed, N. et al. Gene therapy: Comprehensive overview and therapeutic applications. Life Sci 294, (2022).
19. Zu, H. & Gao, D. Non-viral Vectors in Gene Therapy: Recent Development, Challenges, and Prospects. AAPS J 23, (2021).
20. Subhan, M. A., Yalamarty, S. S. K., Filipczak, N., Parveen, F. & Torchilin, V. P. Recent Advances in Tumor Targeting via EPR Effect for Cancer Treatment. J Pers Med 11, 571 (2021).
21. D'Amico, R. S., Aghi, M. K., Vogelbaum, M. A. & Bruce, J. N. Convection-enhanced drug delivery for glioblastoma: a review. J Neurooncol 151, 415 (2021).
22. Ung, T. H., Malone, H., Canoll, P. & Bruce, J. N. Convection-enhanced delivery for glioblastoma: targeted delivery of antitumor therapeutics. CNS Oncol 4, 225 (2015).
23. Gorick, C. M. et al. Applications of focused ultrasound-mediated blood-brain barrier opening. Adv Drug Deliv Rev 191, (2022).
24. Timbie, K. F., Mead, B. P. & Price, R. J. Drug and gene delivery across the blood-brain barrier with focused ultrasound. Journal of Controlled Release (2015) doi:10.1016/j.jconrel.2015.08.059.
25. Bérard, C. et al. Anticancer drug delivery by focused ultrasound-mediated blood-brain/tumor barrier disruption for glioma therapy: From benchside to bedside. Pharmacol Ther 250, 108518 (2023).
26. Curley, C. T. et al. Augmentation of brain tumor interstitial flow via focused ultrasound promotes brain-penetrating nanoparticle dispersion and transfection. Sci Adv 6, (2020).
27. Mead, B. P. et al. Targeted Gene Transfer to the Brain via the Delivery of Brain-Penetrating DNA Nanoparticles with Focused Ultrasound. J Control Release 223, 109 (2016).
28. Bérard, C. et al. Anticancer drug delivery by focused ultrasound-mediated blood-brain/tumor barrier disruption for glioma therapy: From benchside to bedside. Pharmacol Ther 250, 108518 (2023).
29. Lu, V. M. & Niazi, T. N. Clinical Trials of Focused Ultrasound for Brain Tumors. Cancers (Basel) 17, (2025).
30. Banerjee, K. et al. Current Approaches for Glioma Gene Therapy and Virotherapy. Front Mol Neurosci 14, 621831 (2021).
31. Kang, T. et al. Enhancing Glioblastoma-Specific Penetration by Functionalization of Nanoparticles with an Iron-Mimic Peptide Targeting Transferrin/Transferrin Receptor Complex. Mol Pharm 12, 2947-2961 (2015).
32. Kang, T. et al. Synergistic targeting tenascin C and neuropilin-1 for specific penetration of nanoparticles for anti-glioblastoma treatment. Biomaterials 101, 60-75 (2016).
33. Zhao, L. et al. Design and screening of a novel neuropilin-1 targeted penetrating peptide for anti-angiogenic therapy in glioma. Life Sci 270, 119113 (2021).
34. Zhan, C. et al. Cyclic RGD-polyethylene glycol-polyethylenimine for intracranial glioblastoma-targeted gene delivery. Chem Asian J 7, 91-96 (2012).
35. Lin, L. et al. Exploring the in vivo fates of RGD and PEG modified PEI/DNA nanoparticles by optical imaging and optoacoustic imaging. RSC Adv 6, 112552-112561 (2016).
36. Peng, L. et al. Aptamer-conjugated gold nanoparticles targeting epidermal growth factor receptor variant III for the treatment of glioblastoma. Int J Nanomedicine 15, 1363-1372 (2020).
37. Rodríguez-Camacho, A. et al. Glioblastoma Treatment: State-of-the-Art and Future Perspectives. Int J Mol Sci 23, 23 (2022).
38. Hock, N. et al. Thiolated Nanoparticles for Biomedical Applications: Mimicking the Workhorses of Our Body. Advanced Science 9, (2022).
39. Torres, A. G. & Gait, M. J . Exploiting cell surface thiols to enhance cellular uptake. Trends Biotechnol 30, 185-190 (2012).
40. Lee, Y. et al. Visualization of the degradation of a disulfide polymer, linear poly(ethylenimine sulfide), for gene delivery. Bioconjug Chem 18, 13-18 (2007).
41. Peng, Q., Hu, C., Cheng, J., Zhong, Z. & Zhuo, R. Influence of Disulfide Density and Molecular Weight on Disulfide Cross-Linked Polyethylenimine as Gene Vectors. Bioconjug Chem 20, 340-346 (2009).
42. Peng, Q., Zhong, Z. & Zhuo, R. Disulfide Cross-Linked Polyethylenimines (PEI) Prepared via Thiolation of Low Molecular Weight PEI as Highly Efficient Gene Vectors. Bioconjug Chem 19, 499-506 (2008).
43. Lin, G. Q., Yi, W. J., Liu, Q., Yang, X. J. & Zhao, Z. G. Aromatic Thioacetal-Bridged ROS-Responsive Nanoparticles as Novel Gene Delivery Vehicles. Molecules 23, (2018).
44. Muthiah, M. et al. Formulation of glutathione responsive anti-proliferative nanoparticles from thiolated Akt1 siRNA and disulfide-crosslinked PEI for efficient anti-cancer gene therapy. Colloids Surf B Biointerfaces 126, 322-327 (2015).
45. Slezak, A. J. et al. Cysteine-binding adjuvant enhances survival and promotes immune function in a murine model of acute myeloid leukemia. Blood Adv 8, 1747-1759 (2024).
46. Slezak, A. J. et al. Tumor Cell-Surface Binding of Immune Stimulating Polymeric Glyco-Adjuvant via Cysteine-Reactive Pyridyl Disulfide Promotes Antitumor Immunity. ACS Cent Sci 8, 1435-1446 (2022).
47. Kong, X. et al. Specific Tumor Cell Detection by a Metabolically Targeted Aggregation-Induced Emission-Based Gold Nanoprobe. ACS Omega 7, 18073-18084 (2022).
48. Zugates, G. T., Anderson, D. G., Little, S. R., Lawhorn, I. E. B. & Langer, R. Synthesis of poly(beta-amino ester)s with thiol-reactive side chains for DNA delivery. J Am Chem Soc 128, 12726-12734 (2006).
49. Shiels, S. M., Solomon, K. D., Pilia, M., Appleford, M. R. & Ong, J. L. BMP-2 tethered hydroxyapatite for bone tissue regeneration: coating chemistry and osteoblast attachment. J Biomed Mater Res A 100, 3117-3123 (2012).
50. Kane, J. R. et al. Sui generis: gene therapy and delivery systems for the treatment of glioblastoma. Neuro Oncol 17, ii24 (2015).
51. Rong, L., Li, N. & Zhang, Z. Emerging therapies for glioblastoma: current state and future directions. J Exp Clin Cancer Res 41, 142 (2022).
52. Khaitan, D., Reddy, P. L. & Ningaraj, N. Targeting Brain Tumors with Nanomedicines: Overcoming Blood Brain Barrier Challenges. Curr Clin Pharmacol 13, 110-119 (2018).
53. Alberto, M., Paiva-Santos, A. C., Veiga, F. & Pires, P. C. Lipid and Polymeric Nanoparticles: Successful Strategies for Nose-to-Brain Drug Delivery in the Treatment of Depression and Anxiety Disorders. Pharmaceutics 14, (2022).
54. Gorick, C. M. et al. Sonoselective transfection of cerebral vasculature without blood-brain barrier disruption. Proc Natl Acad Sci USA 117, 5644-5654 (2020).
55. Chumakova, O. V. et al. Composition of PLGA and PEI/DNA nanoparticles improves ultrasound-mediated gene delivery in solid tumors in vivo. Cancer Lett 261, 215-225 (2008).
56. Gallops, C., Ziebarth, J. & Wang, Y. A Polymer Physics Perspective on Why PEI Is an Effective Nonviral Gene Delivery Vector. ACS Symposium Series 1350, 1-12 (2020).
57. Mead, B. P. et al. Targeted Gene Transfer to the Brain via the Delivery of Brain-Penetrating DNA Nanoparticles with Focused Ultrasound HHS Public Access. J Control Release 223, 109-117 (2016).
58. Nance, E. et al. Brain-penetrating nanoparticles improve paclitaxel efficacy in malignant glioma following local administration. ACS Nano 8, 10655-10664 (2014).
59. Hock, N. et al. Thiolated Nanoparticles for Biomedical Applications: Mimicking the Workhorses of Our Body. Advanced Science 9, (2022).
60. O'Neill, B. E. et al. Pulsed High Intensity Focused Ultrasound Mediated Nanoparticle Delivery: Mechanisms and Efficacy in Murine Muscle. Ultrasound Med Biol 35, 416-424 (2009).
61. Chen, S. et al. A review of bioeffects induced by focused ultrasound combined with microbubbles on the neurovascular unit. Journal of Cerebral Blood Flow & Metabolism 42, 3 (2021).
62. Durham, P. G. et al. Current clinical investigations of focused ultrasound blood-brain barrier disruption: A review. Neurotherapeutics 21, e00352 (2024).
63. Liu, M., Guo, S. & Stiles, J. K. The emerging role of CXCL10 in cancer (Review). Oncol Lett 2, 583 (2011).
64. Neo, S. Y. & Lundqvist, A. The Multifaceted Roles of CXCL9 Within the Tumor Microenvironment. Adv Exp Med Biol 1231, 45-51 (2020).
65. Groom, J. R. & Luster, A. D. CXCR3 in T cell function. Exp Cell Res 317, 620 (2011).
66. Mastorakos, P. et al. Highly PEGylated DNA Nanoparticles Provide Uniform and Widespread Gene Transfer in the Brain. Adv Healthc Mater 4, 1023-1033 (2015).
67. Negron, K. et al. Widespread gene transfer to malignant gliomas with In vitro-to-In vivo correlation. Journal of Controlled Release 303, 1-11 (2019).
68. Rui, Y. & Green, J. J. Overcoming delivery barriers in immunotherapy for glioblastoma. Drug Deliv Transl Res 11, 2302-2316 (2021).
69. Zhang, Y., Li, Y., Li, S., Zhang, H. & Ma, H. In Situ Monitoring of the Effect of Ultrasound on the Sulfhydryl Groups and Disulfide Bonds of Wheat Gluten. Molecules: A Journal of Synthetic Chemistry and Natural Product Chemistry 23, (2018).
70. Wagner, M. et al. Multiresponsive Polymer Nanoparticles Based on Disulfide Bonds. Macromolecules 54, 2899-2911 (2021).
71. Wei, P., Cornel, E. J. & Du, J. Ultrasound-responsive polymer-based drug delivery systems. Drug Deliv Transl Res 11, 1323 (2021).
72. Chen, X. et al. Functional gene delivery to and across brain vasculature of systemic AAVs with endothelial-specific tropism in rodents and broad tropism in primates. Nature Communications 2023 14:1 14, 1-19 (2023).
73. Krolak, T. et al. A High-Efficiency AAV for Endothelial Cell Transduction Throughout the Central Nervous System. Nature cardiovascular research 1, 389 (2022).

What is claimed is:

1. A method comprising administering to a subject in need thereof, a polymeric nanoparticle comprising free thiol groups.

2. The method of claim 1, wherein the nanoparticle comprises thiol-functionalized polyethylene glycol (PEG-SH).

3. The method of claim 1, wherein the nanoparticle comprises polyethyleneimine (PEI), poly(beta-amino ester) (PBAE), or bioreducible PEI (rPEI).

4. The method of claim 1, wherein the nanoparticle comprises a PEI-g-PEG-SH graft copolymer and a branched PEI polymer.

5. The method of claim 1, wherein nanoparticle is loaded with a therapeutic nucleic acid operably linked to a promoter.

6. The method of claim 5, wherein the promoter is an endothelial cell-specific promoter.

7. The method of claim 6, wherein the promoter is a CD144 promoter.

8. The method of claim 5, wherein the therapeutic nucleic acid encodes a chemokine.

9. The method of claim 8, wherein the chemokine is CXCL9.

10. The method of claim 5, wherein the nanoparticle is administered intravenously.

11. The method of claim 1, further comprising administering microbubbles and focused ultrasound to the subject.

12. The method of claim 11, wherein the nanoparticle is administered between about 30 minutes before and about 30 minutes after the FUS is administered.

13. The method of claim 1, further comprising administering an immunotherapy agent.

14. The method of claim 13, wherein the immunotherapy agent comprises an anti-PD-1 antibody.

15. The method of claim 1, wherein the subject has a cancer.

16. The method of claim 15, wherein the cancer is a glioblastoma.

17. A polymeric nanoparticle comprising free thiol groups, wherein the nanoparticle is loaded with a therapeutic nucleic acid operably linked to a promoter.

18. The polymeric nanoparticle of claim 17, wherein the nanoparticle comprises thiol-functionalized polyethylene glycol (PEG-SH).

19. The polymeric nanoparticle of claim 1, wherein the nanoparticle comprises polyethyleneimine (PEI), poly(beta-amino ester) (PBAE), or bioreducible PEI (rPEI).

20. The polymeric nanoparticle of claim 1, wherein the nanoparticle comprises a PEI-g-PEG-SH graft copolymer and a branched PEI polymer.
